(19) United States
(12) Patent Application Publication
(10) Pub. No.: US 2006/0178394 A1
(43) Pub. Date: Aug. 10, 2006

(54) Pharmaceutical compositions comprising apomorphine for pulmonary inhalation

(76) Inventors: John Nicholas Staniforth, Wiltshire (GB); David Morton, Wiltshire (GB); Michael Tobyn, Wiltshire (GB); Stephen Eason, Wiltshire (GB); Quentin Harmer, Wiltshire (GB); David Ganderton, Wiltshire (GB)

Correspondence Address: DAVIDSON, DAVIDSON & KAPPEL, LLC, 485 SEVENTH AVENUE, 14TH FLOOR, NEW YORK, NY, 10018, US

(21) Appl. No.: 10/552,231

(22) PCT Filed: Apr. 14, 2004
(86) PCT No.: PCT/GB04/01627

§ 371 Date: Apr. 21, 2006

Related U.S. Application Data
(63) Continuation-in-part of application No. 10/413,022, filed on Apr. 14, 2003.
(63) Continuation-in-part of application No. 10/621,964, filed on Jul. 17, 2003.

(51) Int. Cl. A61K 31/473

(52) U.S. Cl. 514/295

(57) ABSTRACT

The present invention relates to inhalable formulations of apomorphine or its pharmaceutically acceptable salts or esters for use in treating sexual dysfunction. The present invention also relates to methods for preparing the apomorphine formulations as well as to methods for treatment of sexual dysfunction using said formulations and inhalers including said formulations. The present invention further relates to the use of apomorphine in the manufacture of a medicament for treating sexual dysfunction.

### BACKGROUND OF THE INVENTION

The term “erectile dysfunction” has been defined by the National Institutes of Health as the inability of the male to attain and maintain erection of the penis sufficient to permit satisfactory sexual intercourse (see J. Am. Med. Assoc., 270(1):83-90 (1993)). Because adequate arterial blood supply is critical for erection, any disorder that impairs blood flow may be implicated in the aetiology of erectile failure. Erectile dysfunction affects millions of men and, although generally regarded as a benign disorder, has a profound impact on their quality of life. It is recognized, however, that in many men psychological desire, orgasmic capacity, and ejaculatory capacity are intact even in the presence of erectile dysfunction.

Aetiological factors for erectile disorders have been categorized as psychogenic or organic in origin.

Psychogenic factors for erectile dysfunction include such processes as depression, anxiety, and relationship problems which can impair erectile functioning by reducing erotic focus or otherwise reducing awareness of sensory experience. This may lead to an inability to initiate or maintain an erection.

Organic factors include those of a neurogenic origin and those of a vasculogenic origin. Neurogenic factors include, for example, lesions of the somatic nervous pathways which may impair reflexogenic erections and interrupt tactile sensations needed to maintain erections, and spinal cord lesions which, depending upon their location and severity, may produce varying degrees of erectile failure. Vasculogenic risk factors include factors which affect blood flow and include cigarette smoking, diabetes mellitus, hypertension, alcohol, vascular disease, high levels of serum cholesterol, low levels of high-density lipoprotein (HDL), and other chronic disease conditions such as arthritis. The Massachusetts Male Aging Study (MMAS, as reported by H. A. Feldman, et al., J. Urol., 151: 54-61 (1994) found, for example, that the age-adjusted probability of complete erectile dysfunction was three times greater in subjects reporting treated diabetes than in those without diabetes. While there is some disagreement as to which of the many aspects of diabetes is the direct cause of erectile dysfunction, vascular disease is most frequently cited.

The MMAS also found a significant correlation between erectile dysfunction and heart disease with two of its associated risk factors, hypertension and low serum high density lipoprotein (HDL). It has been reported that 8-10% of all untreated hypertensive patients are impotent at the time they are diagnosed with hypertension. The association of erectile dysfunction with vascular disease in the literature is strong, with impairments in the hemodynamics of erection demonstrated in patients with myocardial infarction, coronary bypass surgery, cerebrovascular accidents, and peripheral vascular disease. It also found cigarette smoking to be an independent risk factor for vasculogenic erectile dysfunction, with cigarette smoking found to exacerbate the risk of erectile dysfunction associated with cardiovascular diseases.

Females can also suffer from sexual dysfunction. This has been shown to increase with age and is associated with the presence of vascular risk factors and the onset of the menopause. Some of the vascular and muscular mechanisms that contribute to penile erection in males are believed to be similar to the vasculogenic factors in female genital response.

In females, sexual dysfunction can arise from organic causes, from psychogenic causes or from a combination thereof. Female sexual dysfunction includes a failure to attain or maintain vaginal lubrication-swelling responses of sexual excitement until completion of the sexual activity. Organic female sexual dysfunction is known to be related in part to vasculogenic impairment resulting in inadequate blood flow, vaginal engorgement insufficiency and clitoral erection insufficiency.

As described in U.S. Pat. Nos. 5,770,606 and 6,291,471, it is known to treat both psychogenic and organic erectile dysfunction in males with the opioid apomorphine. Two and three milligram sublingual tablets of apomorphine hydrochloride are currently available in Europe for the treatment of male erectile dysfunction under the name Uprima™ (see, e.g., European Public Assessment Report (EPAR) 1945).

Apomorphine is a derivative of morphine, and was first evaluated for use as a pharmacological agent as an emetic in 1869. In the first half of the 20th century, apomorphine was used as a sedative for psychiatric disturbances and as a behaviour-altering agent for alcoholics and addicts. By 1967, the dopaminergic effects of apomorphine were realized, and the compound underwent intensive evaluation for the treatment of Parkinsonism. Since that time, apomorphine has been classified as a selective dopamine receptor agonist that stimulates the central nervous system producing an arousal response manifested by yawning and penile erection in animals and man.

EP 0 689 438A discloses an apomorphine formulation for use in relieving the “off-period” symptoms in patients suffering from Parkinson's disease. The formulation is a dry powder (selected because apomorphine is unstable in an aqueous solution) and it is administered intranasally, for absorption through the nasal mucosa.

In general, there is a prejudice against administering apomorphine by inhalation in the prior art. This is because apomorphine was generally thought to be an irritant compound and it is therefore considered that inhalation of apomorphine would be uncomfortable and unpleasant and should be avoided. For this reason, the dry powder formulations disclosed in EP 0 689 438A comprises particles having a size of between 50 and 100 μm, so that the particles could not accidentally reach the lungs following the described intranasal administration.

WO 00/35457 suggests a method of treating organic erectile dysfunction by the oral administration of a therapeutically effective amount of apomorphine or a pharmaceutically acceptable salt or pro-drug thereof. Apomorphine has the undesirable side effect of causing nausea and it is alleged in this application that it is possible to administer enough apomorphine to achieve the desired therapeutic effect whilst avoiding the nausea. It is suggested that this is possible by administering an amount of apomorphine to obtain plasma concentration levels of apomorphine ranging up to about 5.5 nanograms/millilitre.

WO 01/74358 purports to describe a method for treatment of male erectile dysfunction using an apomorphine formulation. Once again, the invention seeks to achieve the desired therapeutic effect without causing nausea. The patient's plasma concentrations of apomorphine are said to be up to 10 nanograms per millilitre, with less than 15% of patients experiencing emesis. A variety of modes of administration are proposed in WO 01/74358, including inhalation to the lungs. However, the only formulations for inhalation exemplified in WO 01/74358 comprise a solution of apomorphine and sodium metabisulfite in water which is introduced directly into the lungs of a dog via the trachea.

WO 99/38467 purports to describe a method of ameliorating sexual dysfunction in a human female which comprises administering to said human female apomorphine in an amount sufficient to increase intraclitoral blood flow and vaginal wall blood flow on stimulation of said female but less than the amount that induces substantial nausea. In order to achieve this balance, it is suggested that a plasma concentration of apomorphine of no more than about 5.5 nanograms per millilitre be maintained. Sublingual administration of the apomorphine is proposed.

Whilst it has clearly been disclosed in the prior art that apomorphine may be useful in the treatment of sexual dysfunction, the known treatments are still less than ideal. Despite the claims made in the prior art, the treatments regularly cause emesis, even at the apomorphine plasma levels suggested to be free from this side effect. Furthermore, the existing treatments also often suffer from a long delay before the onset of the therapeutic effect. This necessitates an amount of forward planning, where the patient needs to predict when the therapeutic effect is desired and then must administer the dose of apomorphine some time before that.

Whilst in the prior art it has been attempted to keep the dose as low as possible to reduce the concomitant side effects, it has been difficult to strike the necessary balance between efficacy and side effects in the past. However, it has now been found that small doses of apomorphine can be administered by pulmonary inhalation to provide the desired therapeutic effect, whilst avoiding or minimising the side effects usually associated with a therapeutically effective dose of apomorphine.

### SUMMARY OF THE INVENTION

It is an aim of the present invention to provide a treatment for sexual dysfunction which provides a fast onset of the therapeutic effect, which reduces or even avoids the side effects generally associated with the administration of apomorphine, namely nausea and drowsiness, and which is easy to administer.

It has now been discovered that it is possible to administer apomorphine by pulmonary inhalation without irritation being caused. Toxicology studies have been conducted and it was found that inhaled apomorphine was safe in dogs when administered for periods of 28 days at levels at least 12 times the dosage envisaged for achieving the desired therapeutic effects. The studies showed no signs of irritation or other histopathological changes.

It has also been discovered that small particles of apomorphine are rapidly absorbed from the lung and provide an extremely rapid onset of the therapeutic effect of apomorphine. In fact, the onset of the therapeutic effect is significantly faster than that observed following the administration of apomorphine by the available Uprima® sublingual tablets.

Additionally, it has been found that the amount of apomorphine requires to treat sexual dysfunction when said dose is administered by pulmonary inhalation is significantly smaller than the doses provided by the currently available forms of apomorphine for treating sexual dysfunction, such as the Uprima® sublingual tablets and the intranasal apomorphine composition being developed by Nastech.

What is more, it has also been found that administering apomorphine by pulmonary inhalation leads to an extremely beneficial pharmacokinetic profile which provides an exceptionally fast onset of the therapeutic effect with a beneficial duration and fast elimination of the drug from the plasma. This is in contrast to the pharmacokinetics of the Uprima® tablets which exhibit a delayed onset of the therapeutic effect and a long presence of the drug in the plasma, presumably due to the gradual absorption of the drug across the buccal membrane and even a small proportion of the drug being swallowed.

Advantageously, it has also been found that the small dose of apomorphine administered by pulmonary inhalation and/or the plasma concentration profile observed as a result leads to a reduced incidence of side effects generally associated with the administration of apomorphine, including syncope, vomiting and dowsiness.

Finally, it has also been found that apomorphine, which is inherently unstable and readily oxidises, can be formulated for pulmonary inhalation in formulations which exhibit excellent stability over time and which are therefore suited to commercialisation.

In accordance with one aspect of the present invention, new pharmaceutical compositions comprising apomorphine are provided for treating sexual dysfunction by pulmonary inhalation, whilst avoiding or minimising adverse side effects normally associated with the administration of apomorphine.

In accordance with another aspect of the present invention, new methods of treating sexual dysfunction are provided, using new pharmaceutical compositions comprising apomorphine which are administered by pulmonary inhalation. Again, these methods achieve the desired therapeutic effect whilst avoiding the side effects associated with the administration of apomorphine.

The compositions and methods of the present invention also provide a fast onset of the desired therapeutic effect. Furthermore, the compositions and methods of the present invention are also suitable for treating both males and females.

The present invention relates to high performance inhaled delivery of apomorphine, which has a number of significant and unexpected advantages over previously used modes of administration. The mode of administration and the formulations of the present invention make this excellent performance possible.

Apomorphine can exist in a free base form or as an acid addition salt. For the purposes of the present invention apomorphine hydrochloride and the apomorphine free base forms are preferred, but other pharmacologically acceptable forms of apomorphine can also be used. The term “apomorphine” as used herein includes the free base form of this compound as well as the pharmacologically acceptable salts or esters thereof.

In addition to the hydrochloride salt, other acceptable acid addition salts include the hydrobromide, the hydroiodide, the bisulfate, the phosphate, the acid phosphate, the lactate, the citrate, the tartrate, the salicylate, the succinate, the maleate, the gluconate, and the like.

As used herein, the term “pharmaceutically acceptable esters” of apomorphine refers to esters formed with one or both of the hydroxyl functions at positions 10 and 11, and which hydrolyse in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butryates, acrylates and ethylsuccinates.

The free base of apomorphine is particularly attractive in the context of the present invention as it crosses the lung barrier very readily and so it is anticipated that its administration via pulmonary inhalation will exhibit extremely fast onset of the therapeutic effect. Thus, any of the compositions disclosed herein may be formulated using the apomorphine free base.

In accordance with one embodiment of the present invention, the pharmaceutical composition is in the form of a dry powder. Preferably, the dry powder is dispensed by a dry powder inhaler (DPI).

In one embodiment of the present invention, the composition comprises active particles comprising apomorphine, the active particles having a mass median aerodynamic diameter (MMAD) of no more than about 10 μm.

In another embodiment of the present invention, the composition comprises active particles of apomorphine and an additive material which is an anti-adherent material and reduces cohesion between the particles in the composition.

In yet another embodiment of the present invention, the composition comprises active particles comprising apomorphine and carrier particles of an inert excipient material, such as lactose. The carrier particles may have an average particle size of from about 5 to about 1000 μm.

In an alternative embodiment, the composition is a solution or suspension, which is dispensed using a pressurised metered dose inhaler (pMDI). The composition according to this embodiment can comprise the dry powder composition discussed above, mixed with or dissolved in a liquid propellant such as HFA134a or HFA227.

In one embodiment of the present invention, the composition used to treat sexual dysfunction via inhalation comprises a dose of from about 100 μg to about 2400 μg of apomorphine (that is, apomorphine, apomorphine free base, pharmaceutically acceptable salt(s) or ester(s) thereof, based on the weight of the hydrochloride salt). The dose may comprise from about 200 μg to about 1800 μg of said apomorphine, or from about 300 μg to about 1600 μg of said apomorphine, or from about 400 μg to about 1200 μg of said apomorphine. In another embodiment, doses are provided in increments between 400 μg and 1200 μg, based upon the requirements and tolerance of the individual patients. For examples, doses may be provided of about 400, about 500, about 600, about 700, about 800, about 900, about 1000, about 1100 and/or about 1200 μg of said apomorphine.

Where smaller doses are sufficient to achieve the therapeutic effect, for example, when treating female sexual dysfunction, doses may be provided of about 100, about 200, about 300, about 400, about 500 and/or about 600 μg of said apomorphine.

In another embodiment of the present invention, the dose of the powder composition delivers, in vitro, a fine particle dose of from about 100 μg to about 1800 μg of apomorphine (based on the weight of the hydrochloride salt), when measured by a Multistage Liquid Impinger, United States Pharmacopoeia 26, Chapter 601, Apparatus 4 (2003), an Andersen Cascade Impactor or a New Generation Impactor. Preferably, the dose delivers, in vitro, a fine particle dose from about 200 μg to about 1200 μg of said apomorphine, from about 400 μg to about 1000 μg of said apomorphine, from about 400 μg to about 900 μg, or from about 600 μg to about 800 μg of said apomorphine. Alternatively, where less apomorphine is required to achieve the therapeutic effect, for example where female sexual dysfunction is to be treated, the dose preferably delivers, in vitro, a fine particle dose from about 100 μg to about 900 μg of said apomorphine, from about 200 μg to about 600 μg of said apomorphine, from about 200 μg to about 400 μg of said apomorphine.

It has been found that the delivery of apomorphine via pulmonary inhalation is more efficient than delivery by other routes tried in the prior art, such as oral delivery and intranasal. Studies discussed below indicate that a dose of 1200 μg administered by pulmonary inhalation was associated with minor (non-serious) side effects, such as light-headedness, but did not cause serious adverse side effects such as syncope and vomiting. Although not serious, the minor side effects associated with the 1200 μg dose would limit the use of such a dose outside a clinical setting and so greater doses were not investigated. In contrast to these findings, previous studies have not shown that administration of apomorphine by inhalation does not suffer from serious adverse side effects, such as vomiting. Furthermore, studies conducted by Nastech Pharmaceutical Company Inc. into the intranasal delivery of apomorphine indicated that more than 2 mg of apomorphine can be administered in this manner, in a clinical setting, without causing unacceptable side effects.

The dosing efficiency is also indicated by the fact that the clinical effect is observed following administration by inhalation of as little as 400 μg apomorphine. In contrast, the Uprima® sublingual tablets appear to require a minimum of 2 mg to achieve the desired effect.

In some embodiments of the present invention, apomorphine comprises from about 3% to about 80%, from about 5% to about 50%, or from about 15% to about 40% of the powder composition.

In one embodiment of the present invention, a dose includes about 600 μg of apomorphine hydrochloride, and the dose provides, in vivo, a mean Cₘₐₓ of from about 3.5 ng/ml to about 4.9 ng/ml. In another embodiment, a dose includes about 900 μg of apomorphine hydrochloride, and the dose provides, in vivo, a mean Cₘₐₓ of from about 7.4 ng/ml to about 8.8 ng/ml. In yet another embodiment, a dose includes about 1200 μg of apomorphine hydrochloride, and the dose provides, in vivo, a mean Cₘₐₓ of from about 9.2 ng/ml to about 16.2 ng/ml. The Cₘₐₓ for any dose of apomorphine occurs between I and 30 minutes after administration pulmonary inhalation, and preferably after between 1 and 5 minutes. The terminal elimination of the drug is approximately one hour for any dose.

Thus, according to one embodiment of the present invention, a composition comprising apomorphine is provided, wherein the administration of the composition by pulmonary inhalation provides a Cₘₐₓ within 1 to 5 minutes of administration.

In one embodiment, preferably for the treatment of female sexual dysfunction, the Cₘₐₓ is at least 2 ng/ml. In another embodiment, the Cₘₐₓ is at least 7 ng/ml.

In another embodiment of the invention, the administration of the composition by pulmonary inhalation provides a terminal elimination half-life of between 50 and 70 minutes.

In yet another embodiment, the administration of the composition by pulmonary inhalation provides a dose dependent AUC_{0-∞}.

In another embodiment, the administration of the composition by pulmonary inhalation provides a dose dependent AUC₀₋ₜ.

In a further embodiment of the present invention, the administration of the composition by pulmonary inhalation provides a dose dependent Cₘₐₓ.

In accordance with another embodiment of the present invention, a dose of apomorphine is inhaled into the lungs and said dose is sufficient to provide a therapeutic effect in about 10 minutes or less.

In another aspect, the present invention provides unit doses of apomorphine for treating sexual dysfunction. The unit doses comprise the pharmaceutical compositions comprising apomorphine discussed above.

In one embodiment, blisters are provided containing the apomorphine compositions according to the present invention. The blisters are preferably foil blisters and comprise a base having a cavity formed therein, the cavity containing a powder composition, the cavity having an opening which is sealed by a rupturable covering.

The doses and/or drug loaded blisters preferably include from 1 to 5 mg of powder composition, wherein the apomorphine comprises from about 3% to about 80%, from about 5% to about 50%, or from about 15% to about 40% of the powder composition. Where smaller therapeutic doses are required, for example for treating female sexual dysfunction, the apomorphine may comprise from about 3% to about 40%, from about 4% to about 25% or from about 5 to 20% of the powder composition

According to another embodiment of the present invention, a dry powder inhaler device is provided, comprising a composition according to the invention, as described herein.

In a further embodiment, the inhaler is an active inhaler. In yet another embodiment; the inhaler is a breath actuated inhaler device.

In one embodiment, the composition according to the present invention is held in a blister, the contents of which may be dispensed using one of the aforementioned devices. Preferably, the blister is a foil blister.

In another embodiment, the blister comprises polyvinyl chloride or polypropylene in contact with the composition.

In another aspect, the present invention is directed to methods for producing an inhalable aerosol of a powdered apomorphine composition.

In yet another aspect of the present invention, there is provided the use of apomorphine in the manufacture of a medicament for treating sexual dysfunction by pulmonary inhalation.

Although certain of the compositions, methods or treatment, inhalers, blisters, methods for inhaling, and doses have been described above as including a carrier material having a preferred average particle size of from about 40 μm to about 70 μm, it should be appreciated that in accordance with other embodiments, the carrier material in these compositions, methods or treatment, inhalers, blisters, methods for inhaling, and doses can have other average particle size ranges, for example, from about 5 μm to about 1000 μm, from about 10 μm to about 70 μm, from about or from about 20 μm to about 30 μm.

Thus, it is clear from the foregoing that the present invention provides a number of significant advantages over the prior art. In particular, the present invention provides high performance pulmonary delivery of apomorphine. This high performance enables rapid peak blood levels to be achieved and rapid clinical onset of the therapeutic effect. The effect of the pulmonary administration of apomorphine provided by the present invention is consistent and reproducible and this consistency of the high performance administration leads to a reduction in the side effects normally associated with the administration of apomorphine. The consistent high performance also requires a lower total dose compared to that which would be required if other routes of administration were used.

A significant aspect of the present invention is that it allows one to administer much smaller amounts of apomorphine than are used in the prior art whilst achieving greater blood concentrations of apomorphine but with reduced side effects compared to the prior art apomorphine treatments. Indeed, as will be shown below, a dose of 900 μg of apomorphine administered according to the present invention achieves a blood level of apomorphine which is 6 times higher than that achieved by a 4 mg Uprima™ sublingual tablet, but without causing any significant side effects, which is in contrast to the 4 mg tablet which is not marketed because of unacceptable side effect profiles.

### BRIEF DESCRIPTION OF THE DRAWINGS

FIG. 1 shows schematically a preferred inhaler that can be used to deliver the powder formulations according to the present invention.

FIG. 2 shows an asymmetric vortex chamber which may be used in an inhaler device used to dispense the powder formulations of the present invention.

FIG. 3 shows a sectional view of an alternative form of vortex chamber from an asymmetric inhaler.

FIGS. 4A and 4B illustrate the particle size distribution of the lactose of Example 1.

FIGS. 5A and 5B illustrate the particle size distribution of the micronised apomorphine of Example 2.

FIGS. 6A, 6B and 6C show stability data for the 200 μg apomorphine-lactose formulation of Examples 2(a) and 3.

FIGS. 7A and 7B illustrate the results of tests performed on the apomorphine-lactose formulation of Examples 2 and 3.

FIG. 8 illustrates the particle size distribution of the micronised leucine of Example 10.

FIG. 9 illustrates the quality of erection by treatment group for the patients of Example 14.

FIG. 10 illustrates the response rate by treatment group for the patients of Example 14.

FIG. 11 illustrates the onset and duration of effect for the group of patients treated with the placebo in Example 14.

FIG. 12 illustrates the onset and duration of effect for the group of patients treated with 200 μg of apomorphine in Example 14

FIG. 13 illustrates the onset and duration of effect for the group of patients treated with 400 μg apomorphine in Example 14.

FIG. 14 illustrates the onset and duration of effect for the group of patients treated with 800 μg apomorphine in Example 14.

FIG. 15 shows a comparison of the blood levels at 70 minutes after dosing (T₇₀) for each patient for the 400 μg dose and the 800 μg dose, and additionally shows the known mean Cₘₐₓ of 2 mg, 4 mg, and 5 mg Uprima™ sublingual tablets.

FIGS. 16 to 19 show the pharmacokinetic data gathered during the phase I study discussed in Example 15.

FIG. 20 illustrates the amount (in micrograms) in drug that was delivered to each of the 11 components of an ACI in Example 18.

FIG. 21 illustrates the amount (in micrograms) in drug that was delivered to each of the 11 components of an ACI in Example 19.

FIG. 22 shows the through life dose uniformity results of formulation 12A of Example 20.

FIGS. 23A and 23B show the uniformity of delivered dose of the composition according to the present invention from differently filled blisters, as discussed in Example 4.

### DETAILED DESCRIPTION OF THE PREFERRED EMBODIMENTS

The embodiments of the present invention are directed to inhalable formulations of apomorphine or its pharmaceutically acceptable salts or esters for use in treating sexual dysfunction. The embodiments of the present invention also relate to methods for preparing the apomorphine formulations as well as to methods for treatment of sexual dysfunction using said formulations and inhalers including said formulations. The embodiments of the present invention are also directed to the use of apomorphine in the manufacture of a medicament for treating sexual dysfunctions.

The inhalable formulations in accordance with the present invention are preferably administered via a dry powder inhaler (DPI), but can also be administered via a pressurized metered dose inhaler (pMDI), or even via a nebulised system.

In the context of the present invention, the dose (e.g., in micrograms) of apomorphine or its pharmaceutically acceptable salts or esters will be described based upon the weight of the hydrochloride salt (apomorphine hydrochloride). As such, a dose of 100 μg of “apomorphine or its pharmaceutically acceptable salts or esters” means 100 μg of apomorphine hydrochloride, or an equivalent amount of another salt, an ester, or of the base.

Dry Powder Inhaler Formulations

It is known to administer pharmaceutically active agents to a patient by pulmonary administration of a particulate medicament composition which includes the active agent in the form of fine, dry particles (active particles). The size of the active particles is of great importance in determining the site of absorption of the active agent in the lung. In order for the particles be carried deep into the lungs, the particles must be very fine, for example having a mass median aerodynamic diameter (MMAD) of less than 10 μm. Particles having aerodynamic diameters greater than about 10 μm are likely to impact the walls of the throat and generally do not reach the lung. Particles having aerodynamic diameters in the range of about 5 μm to about 2 μm will generally be deposited in the respiratory bronchioles whereas smaller particles having aerodynamic diameters in the range of about 3 to about 0.05 μm are likely to be deposited in the alveoli.

In one embodiment of the present invention, the composition comprises active particles comprising apomorphine, the active particles having an MMAD of no more than about 10 μm. In another embodiment, the active particles have an MMAD of from about 5 μm to about 2 μm. In yet another embodiment, the active particles have aerodynamic diameters in the range of about 3 to about 0.05 μm. In one embodiment of the invention, at least 90% of the particles of apomorphine have a particle size of 5 μm or less.

Particles having a diameter of less than about 10 μm are, however, thermodynamically unstable due to their high surface area to volume ratio, which provides significant excess surface free energy and encourages particles to agglomerate. In the inhaler, agglomeration of small particles and adherence of particles to the walls of the inhaler are problems that result in the active particles leaving the inhaler as large agglomerates or being unable to leave the inhaler and remaining adhered to the interior of the device, or even clogging or blocking the inhaler.

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung. Consequently, it is an aim of the present invention to provide a powder formulation which provides good reproducibility and therefore accurate and predictable dosing.

The metered dose (MD) of a dry powder formulation is the total mass of active agent present in the metered form presented by the inhaler device in question. For example, the MD might be the mass of active agent present in a capsule for a Cyclohaler™, or in a foil blister in an Aspirair™ device.

The emitted dose (ED) is the total mass of the active agent emitted from the device following actuation. It does not include the material left inside or on the surfaces of the device. The ED is measured by collecting the total emitted mass from the device in an apparatus frequently referred to as a dose uniformity sampling apparatus (DUSA), and recovering this by a validated quantitative wet chemical assay.

The fine particle dose (FPD) is the total mass of active agent which is emitted from the device following actuation which is present in an aerodynamic particle size smaller than a defined limit. Where the term fine particle dose or FPD is used herein, the aerodynamic particle size is smaller than 5 μm. The FPD is measured using an impactor or impinger, such as a twin stage impinger (TSI), multi-stage liquid impinger (MSLI), Andersen Cascade Impactor or a Next Generation Impactor (NGI). Each impactor or impinger has a pre-determined aerodynamic particle size collection cut point for each stage. The FPD value is obtained by interpretation of the stage-by-stage active agent recovery quantified by a validated quantitative wet chemical assay where either a simple stage cut is used to determine FPD or a more complex mathematical interpolation of the stage-by-stage deposition is used.

The fine particle fraction (FPF) is normally defined as the FPD divided by the ED and expressed as a percentage. Herein, the term percent fine particle dose (% FPD) is used to mean the percentage of the total metered dose which is delivered with a diameter of not more than 5 μm (i.e., % FPD=100*FPD/total metered dose).

The term “ultrafine particle dose” (UFPD) is used herein to mean the total mass of active material delivered by a device which has a diameter of not more than 3 μm. The term “ultrafine particle fraction” is used herein to mean the percentage of the total amount of active material delivered by a device which has a diameter of not more than 3 μm. The term percent ultrafine particle dose (% UFPD) is used herein to mean the percentage of the total metered dose which is delivered with a diameter of not more than 3 μm (i.e., % UFPD=100*UFPD/total metered dose).

The terms “delivered dose” and “emitted dose” or “ED” are used interchangeably herein. These are measured as set out in the current EP monograph for inhalation products.

“Actuation of an inhaler” refers to the process during which a dose of the powder is removed from its rest position in the inhaler. That step takes place after the powder has been loaded into the inhaler ready for use.

The tendency of fine particles to agglomerate means that the FPF of a given dose can be highly unpredictable and a variable proportion of the fine particles will be administered to the lung, or to the correct part of the lung, as a result. This is observed, for example, in formulations comprising pure drug in fine particle form. Such formulations exhibit poor flow properties and poor FPF.

In an attempt to improve this situation and to provide a consistent FPF and FPD, dry powder formulations often include additive material.

The additive material is intended to reduce the cohesion between particles in the dry powder formulation. It is thought that the additive material interferes with the weak bonding forces between the small particles, helping to keep the particles separated and reducing the adhesion of such particles to one another, to other particles in the formulation if present and to the internal surfaces of the inhaler device. Where agglomerates of particles are formed, the addition of particles of additive material decreases the stability of those agglomerates so that they are more likely to break up in the turbulent air stream created on actuation of the inhaler device, whereupon the particles are expelled from the device and inhaled. As the agglomerates break up, the active particles may return to the form of small individual particles or agglomerates of small numbers of particles which are capable of reaching the lower lung.

In the prior art, dry powder formulations are discussed which include distinct particles of additive material (generally of a size comparable to that of the fine active particles). In some embodiments, the additive material may form a coating, generally a discontinuous coating, on the active particles and/or on any carrier particles.

Preferably, the additive material is an anti-adherent material and it will tend to reduce the cohesion between particles and will also prevent fine particles becoming attached to surfaces within the inhaler device. Advantageously, the additive material is an anti-friction agent or glidant and will give the powder formulation better flow properties in the inhaler. The additive materials used in this way may not necessarily be usually referred to as anti-adherents or anti-friction agents, but they will have the effect of decreasing the cohesion between the particles or improving the flow of the powder. The additive materials are sometimes referred to as force control agents (FCAs) and they usually lead to better dose reproducibility and higher FPFs.

Therefore, an additive material or FCA, as used herein, is a material whose presence on the surface of a particle can modify the adhesive and cohesive surface forces experienced by that particle, in the presence of other particles and in relation to the surfaces that the particles are exposed to. In general, its function is to reduce both the adhesive and cohesive forces.

The reduced tendency of the particles to bond strongly, either to each other or to the device itself, not only reduces powder cohesion and adhesion, but can also promote better flow characteristics. This leads to improvements in the dose reproducibility because it reduces the variation in the amount of powder metered out for each dose and improves the release of the powder from the device. It also increases the likelihood that the active material, which does leave the device, will reach the lower lung of the patient.

It is favourable for unstable agglomerates of particles to be present in the powder when it is in the inhaler device. As indicated above, for a powder to leave an inhaler device efficiently and reproducibly, the particles of such a powder should be large, preferably larger than about 40 μm. Such a powder may be in the form of either individual particles having a size of about 40 μm or larger and/or agglomerates of finer particles, the agglomerates having a size of about 40 μm or larger. The agglomerates formed can have a size of as much as about 1000 μm and, with the addition of the additive material, those agglomerates are more likely to be broken down efficiently in the turbulent airstream created on inhalation. Therefore, the formation of unstable or “soft” agglomerates of particles in the powder may be favoured compared with a powder in which there is substantially no agglomeration. Such unstable agglomerates are stable whilst the powder is inside the device but are then disrupted and broken up when the powder is dispensed.

The reduction in the cohesion and adhesion between the active particles can lead to equivalent performance with reduced agglomerate size, or even with individual particles.

Thus, in another embodiment of the present invention, the composition comprises active particles of apomorphine and an additive material. The additive material may be in the form of particles which tend to adhere to the surfaces of the active particles, as disclosed in WO 97/03649. Alternatively, the additive material may be coated on the surface of the active particles by, for example a co-milling method as disclosed in WO 02/43701.

In certain embodiments of the present invention, the apomorphine formulation is a “carrier free” formulation, which includes only the apomorphine or its pharmaceutically acceptable salts or esters and one or more additive materials. Such carrier free formulations are described in WO 97/03649, the entire disclosure of which is hereby incorporated by reference. In accordance with these embodiments, the powder formulation includes apomorphine or a pharmaceutically acceptable salt or ester thereof and an additive material which includes an anti-adherent material.

The powder includes at least 60% by weight of the apomorphine or a pharmaceutically acceptable salt or ester thereof based on the weight of the powder. Advantageously, the powder comprises at least 70%, more preferably at least 80% by weight of apomorphine or a pharmaceutically acceptable salt or ester thereof based on the weight of the powder. Most advantageously, the powder comprises at least 90%, more preferably at least 95%, more preferably at least 97%, by weight of apomorphine or a pharmaceutically acceptable salt or ester thereof based on the weight of the powder. It is believed that there are physiological benefits in introducing as little powder as possible to the lungs, in particular material other than the active ingredient to be administered to the patient. Therefore, the quantities in which the additive material is added are preferably as small as possible. The most preferred powder, therefore, would comprise more than 99% by weight of apomorphine or a pharmaceutically acceptable salt or ester thereof.

Advantageously, in these “carrier free” formulations, at least 90% by weight of the particles of the powder have a particle size less than 63 μm, preferably less than 30 μm and more preferably less than 10 μm. As indicated above, the size of the apomorphine (or it pharmaceutically acceptable salts) particles of the powder should be within the range of about from 0.1 μm to 5 μm for effective delivery to the lower lung. Where the additive material is in particulate form, it may be advantageous for these additive particles to have a size outside the preferred range for delivery to the lower lung.

It is particularly advantageous for the additive material to comprise an amino acid. Amino acids have been found to give, when present as additive material, high respirable fraction of the active material and also good flow properties of the powder. A preferred amino acid is leucine, in particular L-leucine. Although the L-form of the amino acids is generally preferred, the D- and DL-forms may also be used. The additive material may comprise one or more of any of the following amino acids: leucine, isoleucine, lysine, valine, methionine, cysteine, and phenylalanine. Advantageously, the powder includes at least 80%, preferably at least 90% by weight of apomorphine (or it pharmaceutically acceptable salts) based on the weight of the powder. Advantageously, the powder includes not more than 8%, more advantageously not more than 5% by weight of additive material based on the weight of the powder. As indicated above, in some cases it will be advantageous for the powder to contain about 1% by weight of additive material. The additive material may also (or alternatively) include magnesium stearate or colloidal silicon dioxide.

The additive material or FCA may be provided in an amount from about 0.1% to about 10% by weight, and preferably from about 0.15% to 5%, most preferably from about 0.5% to about 2%. In the context of the present invention, suitable additive materials include, but are not limited to, anti-adherent materials. Additive materials may include, for example, magnesium stearate, leucine, lecithin, and sodium stearyl fumarate, and are described more fully in WO 96/23485, which is hereby incorporated by reference.

When the additive material is micronised leucine or lecithin, it is preferably provided in an amount from about 0.1% to about 10% by weight. Preferably, the additive material comprises from about 3% to about 7%, preferably about 5%, of micronised leucine. Preferably, at least 95% by weight of the micronised leucine has a particle diameter of less than 150 μm, preferably less than 100 μm, and most preferably less than 50 μm. Preferably, the mass median diameter of the micronised leucine is less than 10 μm.

If magnesium stearate or sodium stearyl fumarate is used as the additive material, it is preferably provided in an amount from about 0.05% to about 10%, from about 0.15% to about 5%, from about 0.25% to about 2%, or from about 0.15% to about 0.5%.

In a further attempt to improve extraction of the dry powder from the dispensing device and to provide a consistent FPF and FPD, dry powder formulations often include coarse carrier particles of excipient material mixed with fine particles of active material. Rather than sticking to one another, the fine active particles tend to adhere to the surfaces of the coarse carrier particles whilst in the inhaler device, but are supposed to release and become dispersed upon actuation of the dispensing device and inhalation into the respiratory tract, to give a fine suspension. The carrier particles preferably have MMADs greater than about 90 μm.

The inclusion of coarse carrier particles is also very attractive where very small doses of active agent are dispensed. It is very difficult to accurately and reproducibly dispense very small quantities of powder and small variations in the amount of powder dispensed will mean large variations in the dose of active agent where only very small amounts of the powder is dispensed and the powder comprises mainly active particles. Therefore, the addition of a diluent, in the form of large excipient particles will make dosing more reproducible and accurate.

Carrier particles may be of any acceptable inert excipient material or combination of materials. For example, the carrier particles may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously, the carrier particles comprise a polyol. In particular, the carrier particles may be particles of crystalline sugar, for example mannitol, dextrose or lactose. Preferably, the carrier particles are composed of lactose.

However, a further difficulty which may be encountered when adding coarse carrier particles to a composition of fine active particles is ensuring that the fine particles detach from the surface of the relatively large carrier particles upon actuation of the delivery device.

The step of dispersing the active particles from other active particles and from carrier particles, if present, to form an aerosol of fine active particles for inhalation is significant in determining the proportion of the dose of active material which reaches the desired site of absorption in the lungs. In order to improve the efficiency of that dispersal it is known to include in the composition additive materials of the nature discussed above. Compositions comprising fine active particles carrier particles and additive materials are disclosed in WO 96/23485.

Thus, in one embodiment of the present invention, the composition comprises active particles comprising apomorphine and carrier particles. The carrier particles may have an average particle size of from about 5 to about 1000 μm, from about 4 to about 40 μm, from about 60 to about 200 μm, or from 150 to about 1000 μm. Other useful average particle sizes for carrier particles are about 20 to about 30 μm or from about 40 to about 70 μm.

The composition comprising apomorphine and carrier particles may further include additive material. The additive material may be in the form of particles which tend to adhere to the surfaces of the active particles, as disclosed in WO 97/03649. Alternatively, the additive material may be coated on the surface of the active particles by, for example a co-milling method as disclosed in WO 02/43701 or on the surfaces of the carrier particles, as disclosed in WO 02/00197.

In a dry powder inhaler, the dose to be administered is stored in the form of a non-pressurized dry powder and, on actuation of the inhaler, the particles of the powder are inhaled by the patient. Dry powder inhalers can be “passive” devices in which the patient's breath is the only source of gas which provides a motive force in the device. Examples of “passive” dry powder inhaler devices include the Rotahaler and Diskhaler (GlaxoSmithKline) and the Turbohaler (Astra-Draco) and Novolizer™ (Viatris GmbH). Alternatively, “active” devices may be used, in which a source of compressed gas or alternative energy source is used. Examples of suitable active devices include Aspirair™ (Vectura Ltd) and the active inhaler device produced by Nektar Therapeutics (as covered by U.S. Pat. No. 6,257,233).

Particularly preferred “active” dry powder inhalers are referred to herein as Aspriair inhalers and are described in more detail in WO 01/00262, WO 02/07805, WO 02/89880 and WO 02/89881, the contents of which are hereby incorporated by reference. It should be appreciated, however, that the compositions of the present invention can be administered with either passive or active inhaler devices.

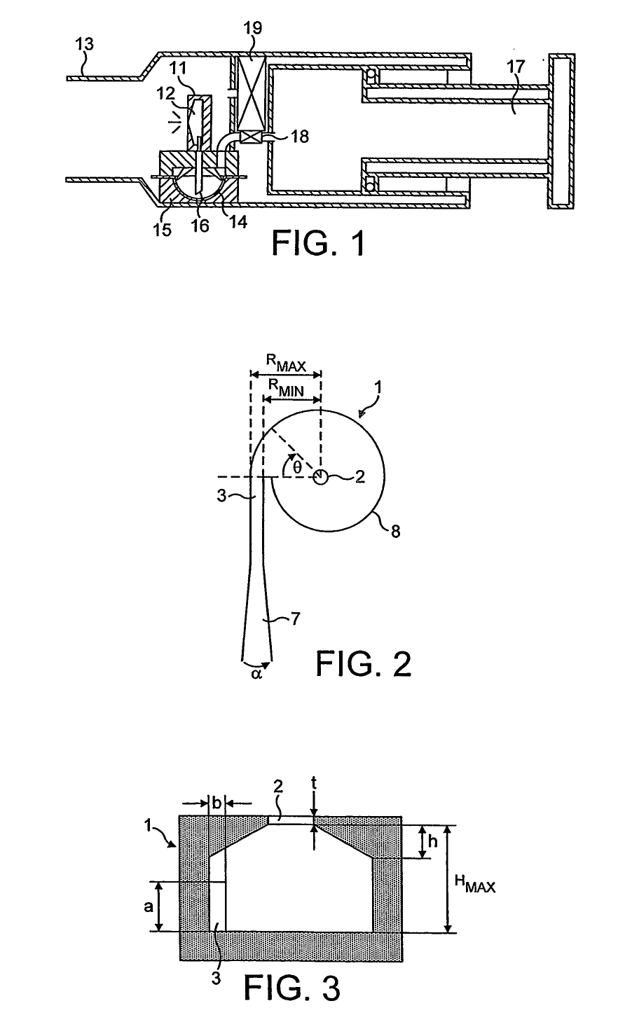

FIG. 1 shows schematically a preferred inhaler that can be used to deliver the powder formulations described above to a patient. Inhalers of this type are described in detail in WO 02/089880 and WO 02/089881.

Referring to FIGS. 1 and 2, the inhaler comprises a vortex nozzle 11 including a vortex chamber 12 and having an exit port and an inlet port for generating an aerosol of the powder formulation. The vortex chamber is located in a mouthpiece 13 through which the user inhales to use the inhaler. Air passages (not shown) may be defined between the vortex chamber and the mouthpiece so that the user is able to inhale air in addition to the powdered medicament.

The powder formulation is stored in a blister 14 defined by a support and a pierceable foil lid. A blister holder 15 holds the blister in place. As shown, the support has a cavity formed therein for holding the powder formulation. The open end of the cavity is sealed by the lid. An air inlet conduit of the vortex chamber terminates in a piercing head 16 which pierces the pierceable foil lid. A reservoir 17 is connected to the blister via a passage. An air supply, preferably a manually operated pump or a canister of pressurized gas or propellant, charges the reservoir with a gas (e.g., air, in this example) to a predetermined pressure (e.g. 1.5 bar). In a preferred embodiment the reservoir comprises a piston received in a cylinder defining a reservoir chamber. The piston is pushed into the cylinder to reduce the volume of the chamber and pressurize the charge of gas.

When the user inhales, a valve 18 is opened by a breath-actuated mechanism 19, forcing air from the pressurized air reservoir through the blister where the powdered formulation is entrained in the air flow. The air flow transports the powder formulation to the vortex chamber 12, where a rotating vortex of powder formulation and air is created between the inlet port and the outlet port. Rather than passing through the vortex chamber in a continuous manner, the powdered formulation entrained in the airflow enters the vortex chamber in a very short time (typically less than 0.3 seconds and preferably less than 20 milliseconds) and, in the case of a pure drug formulation (i.e., no carrier), a portion of the powder formulation sticks to the walls of the vortex chamber. This powder is subsequently aerosolized by the high shear forces present in the boundary layer adjacent to the powder. The action of the vortex deagglomerates the particles of powder formulation, or in the case of a formulation comprising a drug and a carrier, strips the drug from the carrier, so that an aerosol of powdered formulation exits the vortex chamber via the exit port. The aerosol is inhaled by the user through the mouthpiece.

The vortex chamber can be considered to perform two functions: deagglomeration, the breaking up of clusters of particles into individual, respirable particles; and filtration, preferentially allowing particles below a certain size to escape more easily from the exit port. Deagglomeration breaks up cohesive clusters of powdered formulation into respirable particles, and filtration increases the residence time of the clusters in the vortex chamber to allow more time for them to be deagglomerated. Deagglomeration can be achieved by turbulence and by creating high shear forces due to velocity gradients in the airflow in the vortex chamber. The velocity gradients are highest in the boundary layer close to the walls of the vortex chamber.

The vortex chamber is in the form of a substantially cylindrical chamber. Advantageously, the vortex chamber has an asymmetric shape. In the embodiment shown in FIGS. 2 and 3, the wall 8 of the vortex chamber is in the form of a spiral or scroll. The inlet port 3 is substantially tangential to the perimeter of the vortex chamber 1 and the exit port 2 is generally concentric with the axis of the vortex chamber 1. Thus, gas enters the vortex chamber 1 tangentially via the inlet port 3 and exits axially via the exit port 2. The radius R of the vortex chamber 1 measured from the center of the exit port 2 decreases smoothly from a maximum radius Rₘₐₓ at the inlet port to a minimum radius Rₘᵢₙ. Thus, the radius R at an angle θ (theta) from the position of the inlet port 3 is given by R═Rₘₐₓ(1−θk/2 pi), where k=(Rₘₐₓ—Rₘᵢₙ)/Rₘₐₓ. The effective radius of the vortex chamber 1 decreases as the air flow and entrained particles of medicament circulate around the chamber. In this way, the effective cross-sectional area of the vortex chamber 1 experienced by the air flow decreases, so that the air flow is accelerated and there is reduced deposition of the entrained particles of medicament. In addition, when the flow of air has gone through 2 pi radians (360°), the air flow is parallel to the incoming airflow through the inlet port 3, so that there is a reduction in the turbulence caused by the colliding flows which helps reduce fluid losses in the vortex.

Between the inlet port 3 and the exit port 2 a vortex is created in which shear forces are generated to deagglomerate the particles of the powdered formulation. The length of the exit port 2 is preferably as short as possible to reduce the possibility of deposition of the drug on the walls of the exit port. FIG. 3 shows the general form of the vortex chamber of the inhaler of FIG. 2. The geometry of the vortex chamber is defined by the dimensions listed in the table below. The preferred values of these dimension are also listed in the table. It should be noted that the preferred value of the height h of the conical part of the chamber is 0 mm, because it has been found that the vortex chamber functions most effectively when the top (roof) of the chamber is flat.

| | | Preferred |
|---|---|---|
| Dimension | | Value |
| Rₘₐₓ | Maximum radius of chamber | 2.8 mm |
| Rₘᵢₙ | Minimum radius of chamber | 2.0 mm |
| Hₘₐₓ | Maximum height of chamber | 1.6 mm |
| h | Height of conical part of chamber | 0.0 mm |
| Dₑ | Diameter of exit port | 0.7 mm |
| t | Length of exit port | 0.3 mm |
| a | Height of inlet port | 1.1 mm |
| b | Width of inlet port | 0.5 mm |
| α | Taper angle of inlet conduit | 9°, then 2° |

The ratio of the diameter of the chamber 1 to the diameter of the exit port 2 has a strong influence on the aerosolizing performance of the nozzle. For the asymmetric nozzle of FIG. 2, the diameter is defined as (Rₘₐₓ+Rₘᵢₙ). The ratio is between 4 and 12 and preferably between 6 and 8. In the preferred embodiment of FIGS. 2 and 3, the ratio is 6.9.

In the embodiment shown, the vortex chamber is machined from polyetheretherketone (PEEK), acrylic, or brass, although a wide range of alternative materials is possible. Advantageously for high volume manufacture the vortex chamber is injection moulded from a polymer. Suitable materials include but are not limited to polycarbonate, acrylonitrile butadiene styrene (ABS), polyamides, polystyrenes, polybutylene terphthalate (PBT) and polyolefins including polypropylene and polyethylene terephthalate (PET).

The inhaler in accordance with embodiments of the invention is able to generate a relatively slow moving aerosol with a high fine particle fraction. The inhaler is capable of providing complete and repeatable aerosolisation of a measured dose of powdered drug and of delivering the aerosolised dose into the patient's inspiratory flow at a velocity less than or substantially equal to the velocity of the inspiratory flow, thereby reducing deposition by impaction in the patient's mouth. Furthermore, the efficient aerosolising system allows for a simple, small and low cost device, because the energy used to create the aerosol is small. The fluid energy required to create the aerosol can be defined as the integral over time of the pressure multiplied by the flow rate. This is typically less than 5 joules and can be as low as 3 joules.

In certain embodiments of the present invention, the powder composition is such that a fine particle fraction of at least 35% is generated on actuation of the inhaler device. It is particularly preferred that the fine particle fraction be greater than or equal to 45%, 50% or 60%. Preferably, the fine particle fraction is at least 70%, and most preferably at least 80%. In one embodiment, this powder comprises apomorphine in combination with a carrier material.

Most preferably, the inhaler device used to dispense the powder composition is an active inhaler device, the arrangement being such that a fine particle fraction of at least 35%, preferably at least 50%, even more preferably at least 60%, even more preferably at least 70%, and most preferably at least 80% is generated on actuation of the inhaler device. As an active device does not depend on the patient's inhalation for aerosolising the dose, the delivery of the dose is more repeatable than is observed using passive inhaler devices.

In accordance with another embodiment of the present invention, the dose of apomorphine or a pharmaceutically acceptable salt or ester thereof is defined in terms of the fine particle dose of the administered dose. The percentage of the apomorphine in the dose which will reach the lung (the % FPD) is dependent on the formulation used and on the inhaler used. As such, a 1000 μg dose of apomorphine hydrochloride will deliver 350 μg of apomorphine to the lung of a patient if a % FPD of 35% is achieved, whilst the same dose will deliver 600 μg of apomorphine to the lung of a patient if a % FPD of 60% is achieved, or 700 μg if the % FPD is 70%, as anticipated in the present invention. As such, it is appropriate to define the dose of apomorphine in terms of the FPD of the formulation and inhaler used, as measured by a Multistage Liquid Impinger or an Anderson Cascade Impactor.

As such, in accordance with another embodiment of the present invention, a method for treating sexual dysfunction via inhalation is provided which comprises inhaling a dose of a powder composition into the lungs of a patient, the dose of the powder composition delivering, in vitro, a fine particle dose of from about 100 μg to about 1800 μg of apomorphine (based on the weight of the hydrochloride salt), when measured by a Multistage Liquid Impinger, United States Pharmacopoeia 26, Chapter 601, Apparatus 4 (2003), an Andersen Cascade Impactor or a New Generation Impactor. Preferably, the dose delivers, in vitro, a fine particle dose of said apomorphine of from about 200 μg to about 1200 μg, from about 400 μg to about 1000 μg, from about 400 μg to about 900 μg, or from about 600 μg to about 800 μg. Alternatively, where less apomorphine is required to achieve the therapeutic effect, for example where female sexual dysfunction is to be treated, the dose preferably delivers, in vitro, a fine particle dose from about 100 μg to about 900 μg of said apomorphine, from about 200 μg to about 600 μg of said apomorphine, from about 200 μg to about 400 μg of said apomorphine.

The dose of apomorphine (which includes apomorphine free base or pharmaceutically acceptable salt(s) or ester(s) of apomorphine, based on the weight of the hydrochloride salt), defined in the manner above in connection with the Multistage Liquid Impinger, can similarly be used in connection with the blisters, inhalers, and compositions described herein.

In addition to the fine particle fraction, another parameter of interest is the ultrafine particle fraction defined above. Although particles having a diameter of less than 5 μm (corresponding to the FPF) are suitable for local delivery to the lungs, it is believed that for systemic delivery, even finer particles are needed, because the drug must reach the alveoli to be absorbed into the bloodstream. As such, it is particularly preferred that the formulations and devices in accordance with the present invention be sufficient to provide an ultrafine particle fraction of at least about 50%, more preferably at least about 60% and most preferably at least about 70%.

Preferably, at least 90% by weight of the active material has a particle size of not more than 10 μm, most preferably not more than 5 μm. The particles therefore give a good suspension on actuation of the inhaler.

According to an embodiment of the present invention, an active inhaler device may be used to dispense the apomorphine dry powder formulations, in order to ensure that the best fine particle fraction and fine particle dose is achieved and, very importantly, that this is achieved consistently. Preferably, the inhaler device includes a breath triggering means such that the delivery of the dose is triggered by the onset of the patient's inhalation. This means that the patient does not need to coordinate their inhalation with the actuation of the inhaler device and that the dose can be delivered at the optimum point in the inspiratory flow. Such devices are commonly referred to as “breath actuated”.

In embodiments of the present invention which utilize conventional inhalers, such as the Rotohaler and Diskhaler described above, the particle size of the carrier particles may range from about 10 to about 1000 μm. In certain of these embodiments, the particle size of the carrier particles may range from about 20 μm to about 120 μm. In certain other ones of these embodiments, the size of at least 90% by weight of the carrier particles is less than 1000 μm and preferably lies between 60 μm and 1000 μm. The relatively large size of these carrier particles gives good flow and entrainment characteristics.

In these embodiments, the powder may also contain fine particles of an excipient material, which may for example be a material such as one of those mentioned above as being suitable for use as a carrier material, especially a crystalline sugar such as dextrose or lactose. The fine excipient material may be of the same or a different material from the carrier particles, where both are present. The particle size of the fine excipient material will generally not exceed 30 μm, and preferably does not exceed 20 μm.

The powders may also be formulated with additional excipients to aid delivery and release. For example, as discussed above, powder compositions may be formulated with relatively large carrier particles, for example those having a mass median aerodynamic diameter of greater than 90 μm, which aid the flow properties of the powder. Alternatively, hydrophobic microparticles may be dispersed within a carrier material. For example, the hydrophobic microparticles may be dispersed within a polysaccharide or polymeric matrix, with the overall composition formulated as microparticles for direct delivery to the lung. The polysaccharide or polymer act as a further barrier to the immediate release of the active agent. This may further aid the controlled release process. An example of a suitable polysaccharide is xanthan gum, whilst suitable polymeric materials include polylactic acid, polyglycolic acid, and the like. Preferred hydrophobic materials include solid state fatty acids such as oleic acid, lauric acid, palmitic acid, stearic acid, erucic acid, behenic acid, or derivatives (such as esters and salts) thereof. Specific examples of such materials include phosphatidylcholines, phosphatidylglycerols and other examples of natural and synthetic lung surfactants. Particularly preferred materials include metal stearates, in particular magnesium stearate, which has been approved for delivery via the lung.

Large carrier particles are particularly useful when they are included in compositions which are to be dispensed using a passive inhaler device, such as the Diskhaler and Rotahaler devices discussed above. These devices do not create high turbulence within the device upon actuation and so the presence of the carrier particles is beneficial as they have a beneficial effect on the flow properties of the powder, making it easier to extract the powder from the blister or capsule within which it is stored.

In some circumstances, the powder for inhalation may be prepared by mixing the components of the powder together. For example, the powder may be prepared by mixing together particles of active material and lactose.

In embodiments of the present invention which utilize an active inhaler, for example an Aspirair inhaler as described above, the carrier particles are preferably between 5 and 100 μm, and may be between 40 and 70 μm in diameter or between 20 and 30 μm in diameter. The desired particle size can be achieved for example, by sieving the excipient. For a desired particle size range of between 40 and 70 μm, the material may be sieved through screens of 45 μm and 63 μm, thereby excluding particles that pass through the 45 μm screen, and excluding particles that do not pass through the 63 μm screen. Most preferably, the excipient is lactose.

Preferably, at least 90%, and most preferably at least 99%, of the apomorphine particles are 5 μm or less in diameter. As detailed below, such a formulation, when administered via the preferred active inhalers, can provide a fine particle fraction in excess of about 80%, and an ultrafine particle fraction in excess of about 70%.

In such formulations where the dispensing device creates high turbulence within the device upon actuation, the powder does not need to include large carrier particles to enhance the flow properties of the powder. The device is capable of extracting powders even if they have poor flow properties and so the diluent material used in such formulations can have a smaller particle size. In one embodiment, the particles of excipient material may even be 10 μm in diameter or less.

The dry powder inhaler devices in which the powder compositions of the present invention will commonly be used include “single dose” devices, for example the Rotahaler™ and the Spinhaler™ in which individual doses of the powder composition are introduced into the device in, for example, single dose capsules or blisters, and also multiple dose devices, for example the Turbohaler™ in which, on actuation of the inhaler, one dose of the powder is removed from a reservoir of the powder material contained in the device.

As already mentioned, in the case of certain powders, an active inhaler device offers advantages in that a higher fine particle fraction and a more consistent dose to dose repeatability will be obtainable than if other forms of device were used. Such devices include, for example, the Aspirair™ or the Nektar Therapeutics active inhaler device, and may be breath actuated devices of the kind in which generation of an aerosolised cloud of powder is triggered by inhalation of the patient.

Where present, the amount of carrier particles may be up to 99%, up to 95%, up to 90%, up to 80% or up to 50% by weight based on the total weight of the powder. The amount of any fine excipient material, if present, may be up to 50% and advantageously up to 30%, especially up to 20%, by weight, based on the total weight of the powder.

Where reference is made to particle size of particles of the powder, it is to be understood, unless indicated to the contrary, that the particle size is the volume weighted particle size. The particle size may be calculated by a laser diffraction method. Where the particle also includes an additive material on the surface of the particle, advantageously the particle size of the coated particles is also within the preferred size ranges indicated for the uncoated particles.

While it is clearly desirable for as large a proportion as possible of the particles of active material to be delivered to the deep lung, it is usually preferable for as little as possible of the other components to penetrate the deep lung. Therefore, powders generally include particles of an active material, and carrier particles for carrying the particles of active material.

As described in WO 01/82906, an additive material may also be provided in a dose which indicates to the patient that the dose has been administered. The additive material, referred to below as indicator material, may be present in the powder as formulated for the dry powder inhaler, or be present in a separate form, such as in a separate location within the inhaler such that the additive becomes entrained in the airflow generated on inhalation simultaneously or sequentially with the powder containing the active material.

In some circumstances, for example, where any carrier particles and/or any fine excipient material present is of a material itself capable of inducing a sensation in the oropharyngeal region, the carrier particles and/or the fine excipient material can constitute the indicator material. For example, the carrier particles and/or any fine particle excipient may comprise mannitol. Another suitable indicator material is menthol.

As discussed above, in an embodiment of the present invention, an inhalable powder composition is provided which includes apomorphine in combination with a carrier material. An example of a suitable apomorphine ester is diisobutyryl apomorphine. Alternatively, the apomorphine comprises apomorphine hydrochloride or the apomorphine is in the free base form.

In any event, the apomorphine is provided in an amount from about 200 μg to about 1800 μg of said apomorphine, or from about 300 μg to about 1600 μg of said apomorphine, or form about 400 to about 1200 μg of said apomorphine. In another embodiment, doses are provided in increments between 400 and 1200 μg, based upon the requirements and tolerance of the individual patients. For examples, doses may be provided of about 400, about 500, about 600, about 700, about 800, about 900, about 1000, about 1100 and/or about 1200 μg of said apomorphine. Where smaller doses are sufficient to achieve the therapeutic effect, for example, when treating female sexual dysfunction, doses may be provided of about 100, about 200, about 300, about 400, about 500 and/or about 600 μg of said apomorphine.

These powder compositions, when inhaled, preferably exhibit a time to therapeutic effect of less than 15 minutes, preferably less than about 10 minutes, and most preferably less than about 9 minutes. This is supported by the pharmacokinetic data discussed in greater detail below. The data indicates that the Cₘₐₓ was achieved after between 1 and 3 minutes in all subjects except one and for all of the doses of apomorphine tested. Elimination of the drug from the plasma is relatively rapid, with a terminal half-life of approximately 60 minutes being observed for all doses tested in the pharmacokinetic studies. Such a fast elimination of the drug from the plasma is advantageous because apomorphine is known to have side effects such as drowsiness which may impair the patient from performing certain tasks, such as operating a motor vehicle or heavy equipment.

Additionally, dose proportionality was also demonstrated for Cₘₐₓ, AUC_{0-∞} and AUC₀₋ₜ.

In certain embodiments of the present invention, each dose is stored in a foil “blister” of a blister pack. Apomorphine is susceptible to oxidation, and, as such, it is important to prevent (or substantially limit) oxidation of the apomorphine prior to administration. In accordance with the embodiments of the present invention which utilize foil blisters, exposure of the formulation to air prior to administration (and unacceptable oxidation of the apomorphine) is prevented by storing each dose in a sealed foil blister. The sealed foil blister will generally be sufficient to protect the apomorphine from oxidation, however, in certain climates, such as those found in parts of the world like the Far East, hydrolysis is a potential problem and hydrolysis is further prevented (or limited) by placing a plurality of blisters into a further sealed container, such as a sealed bag made, for example of a foil such as aluminium foil. Further mechanical protection may also be desirable, to protect the sealed blisters from damage during storage and transportation, etc. The use of the sealed foil blisters (and optional sealed bags and/or other protective packaging) eliminates any need to include anti-oxidants in the formulation.

The apomorphine dry powder compositions according to the present invention were transferred into foil blisters for the experiments discussed below. The blisters consist of a base and a lid. The base material is a laminate comprising a polymer layer in contact with the drug, a soft tempered aluminium layer and an external polymer layer. The aluminium provides the moisture and oxygen barrier, whilst the polymer provides a relatively inert layer in contact with the drug. Soft tempered aluminium is ductile so that it can be “cold formed” into a blister shape. It is typically 45-47 μm thick. The outer polymer layer provides additional strength to the laminate. The lid material is a laminate comprising a heat seal lacquer, a hard rolled aluminium layer (typically 20-30 μm thick) and an external polymer layer. The heat seal lacquer bonds to the polymer layer of the base foil laminate during heat sealing. The aluminium layer is hard rolled to facilitate piercing. Materials for the polymer layer in contact with the drug include polyvinyl chloride (PVC), polypropylene (PP) and polyethylene (PE). The external polymer layer on the base foil is typically oriented polyamide (oPA).

Pressurized Metered Dose Inhaler Formulations

Pressurized metered dose inhalers (pMDI) typically have two components: a canister component in which the drug particles (in this case apomorphine or its pharmaceutically acceptable salts or esters) are stored under pressure in a suspension or solution form and a receptacle component used to hold and actuate the canister. Typically, a canister will contain multiple doses of the formulation, although it is possible to have single dose canisters as well. The canister component typically includes a valved outlet from which the contents of the canister can be discharged. Aerosol medication is dispensed from the pMDI by applying a force on the canister component to push it into the receptacle component thereby opening the valved outlet and causing the medication particles to be conveyed from the valved outlet through the receptacle component and discharged from an outlet of the receptacle component. Upon discharge from the canister, the medication particles are “atomised” forming an aerosol.

It is intended that the patient coordinate the discharge of aerosolised medication with his or her inhalation so that the medication particles are entrained in the patient's inspiratory flow and conveyed to the lungs.

Typically, pMDIs use propellants to pressurize the contents of the canister and to propel the medication particles out of the outlet of the receptacle component. In pMDI inhalers, the formulation is provided in liquid form, and resides within the container along with the propellant. The propellant can take a variety of forms. For example, the propellant can comprise a compressed gas or a liquefied gas.

Suitable propellants include CFC (chlorofluorocarbon) propellants such as CFC 11 and CFC 12, as well as HFA (Hydrofluoroalkane) propellants such as HFA134a and HFA227. One or more propellants may be used in a given formulation.

In order to better coordinate actuation of the inhaler with inhalation, a breath actuated valve system may be used. Such systems are available, for example, from Baker Norton and 3M. To use such a device, the patient “primes” the device, and then the dose is automatically fired when the patient inhales.

In accordance with certain embodiments of the present invention, a pMDI formulation is used to deliver the apomorphine to the lungs of the patient. The apomorphine is provided in an amount from about 200 μg to about 1800 μg of said apomorphine, or from about 300 μg to about 1600 μg of said apomorphine, or form about 400 μg to about 1200 μg of said apomorphine. In another embodiment, doses are provided in increments between 400 μg and 1200 μg, based upon the requirements and tolerance of the individual patients. For examples, doses may be provided of about 400, about 500, about 600, about 700, about 800, about 900, about 1000, about 1100 and/or about 1200 μg of said apomorphine.

Where smaller doses are sufficient to achieve the therapeutic effect, for example, when treating female sexual dysfunction, doses may be provided of about 100, about 200, about 300, about 400, about 500 and/or about 600 μg of said apomorphine.

In certain embodiments, the pMDI formulation is either a “suspension” type formulation or a “solution” type formulation, each using a liquefied gas as the propellant. It is believed that the in vivo affect of pMDI formulations will be similar to those of the DPI formulations described above, in terms of time to therapeutic effect, and duration of therapeutic effect.

Solution pMDI

Of pMDI technologies, solution pMDIs are believed to be the most appropriate for systemic lung delivery as they offer the finest mist, and can be more easily optimised through modifications to the device. Recently developed valves (e.g. available from Bespak) also offer payload increases over current systems, meaning that larger systemic doses can potentially be delivered in solution pMDIs than in suspension type pMDIs.

Solution pMDI techniques can be used to prepare formulations for delivery of apomorphine esters (for example, diisobutyryl apomorphine) with HFA propellants.

However, conventional solution pMDI techniques are not believed to be appropriate for the delivery of apomorphine or its pharmaceutically acceptable salts with HFA propellants. Specifically, apomorphine base is too unstable to be formulated using current approaches and apomorphine salts are too polar to be formulated as solutions in HFA propellants. For example, apomorphine HCl requires at least 50% ethanol for suitable or acceptable solubility in these systems, and such systems would neither be technologically acceptable or likely to be accepted by patients. Even with such a system, a solution concentration of <25 μg/dose is achieved, which is well below the effective doses described above in connection with Dry Powder Inhalers.

In the past, formulators sought to minimize the amount of water present in a pMDI solution because water was known to reduce the fine particle fraction of the formulation (e.g., as reported in WO 02/030499) and/or to reduce the stability of the formulation (e.g., as reported in WO 01/89616).

In accordance with an embodiment of the present invention, a pMDI solution including apomorphine or its pharmaceutically acceptable salts is surprisingly provided through the deliberate addition of water to the system. Specifically, it is believed that a suitable pMDI solution can be obtained by adding the apomorphine or its pharmaceutically acceptable salts to a propellant solution which includes from about 50% to about 98% w/w HFA134a (1,1,1,2-tetrafluoroethane) and/or HFA227 (1,1,1,2,3,3,3-heptafluoropropane), from about 2% to about 10% w/w water, and from about 0% to about 47% w/w ethanol. Preferably, the water is provided in an amount from greater than 5% to about 10% w/w. With regard to ethanol, it is preferably provided in an amount from about 12% to about 40% w/w. Preferably, a 12 ml solution would include about 170 mg of apomorphine hydrochloride in addition to the HFA134a, water and/or ethanol. A 3M coated (DUPONT 3200 200) canister can be used as the canister for the inhaler.

Suspension pMDI

Suspension pMDIs can also be used to deliver apomorphine or its pharmaceutically acceptable salts to the lungs. However, suspension pMDIs have a number of disadvantages. For example, suspension pMDIs generally deliver lower doses than solution pMDIs and are prone to other issues related to suspensions, e.g., dose inconsistencies, valve blockage, and suspension instabilities (e.g., settling). For these reasons, and others, suspension pMDIs tend to be much more complex to formulate and manufacture than solution pMDIs.

In accordance with one embodiment of the present invention, a suspension pMDI for apomorphine or its pharmaceutically acceptable salts is provided. Preferably, the propellant of the suspension pMDI is a blend of two commercially available HFA propellants, most preferably about 60% HFA227 (1,1,1,2,3,3,3-heptafluoropropane) and about 40% HFA134a (1,1,1,2-tetrafluoroethane). This approach showed initial physical stability (due to density matching) without addition of further excipients. This is suggestive that such systems are readily capable of manufacture, although other excipients may be added at low levels to improve pharmaceutical elegance. For example, blends of about 60% HFA227 and about 40% HFA134a were prepared with apomorphine hydrochloride in a 3M coated (Dupont 3200 200) canister with a Bespak BK630 series 0.22 mm actuator. The results of these experiments are discussed below in connection with Example 16.

Nebulised Systems

Another possible method of administration is via a nebulised system. Such systems include conventional ultrasonic nebulised systems and jet nebulised systems, as well as recently introduced handheld devices such as the Respimat (available from Boehringer Ingelheim) or the AERx (available from Aradigm). In such a system, the apomorphine or a pharmaceutically acceptable salt or ester thereof could be stabilized in a sterile aqueous solution, for example, with antioxidants such as sodium metabisulfite. The doses would be similar to those described above, adjusted to take into consideration the lower percentage of apomorphine that will reach the lung in a nebulised system. Although these systems can be used, they are clearly inferior to the DPI systems described above, both in terms of efficiency and convenience of use.

### EXAMPLES

Various examples illustrating the invention are discussed below. Unless otherwise stated, the inhaler device used in the examples was an Aspirair prototype inhaler made by Vectura Limited.

### Example 1

### Preparation of Lactose

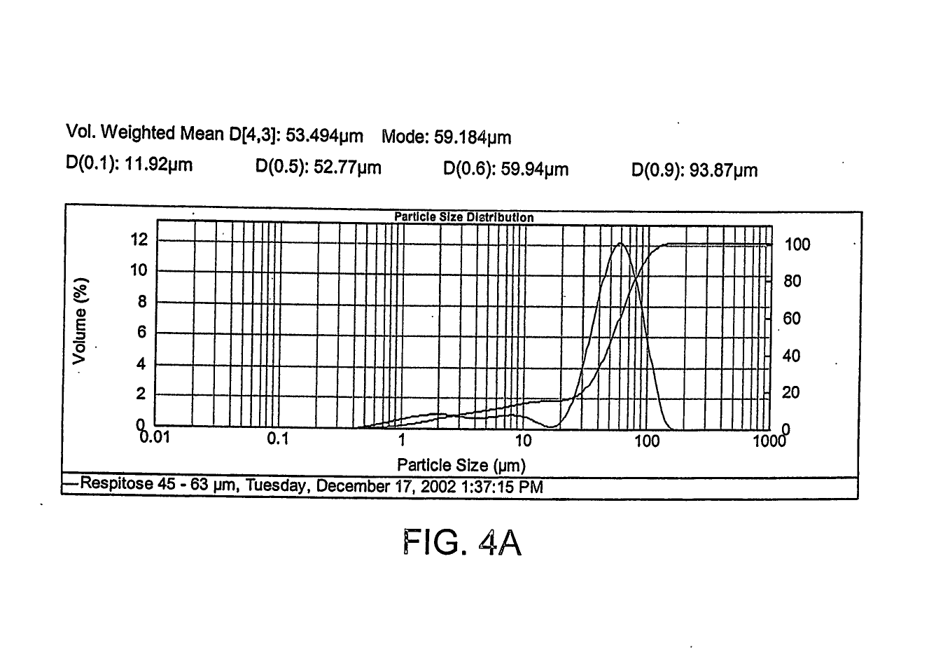

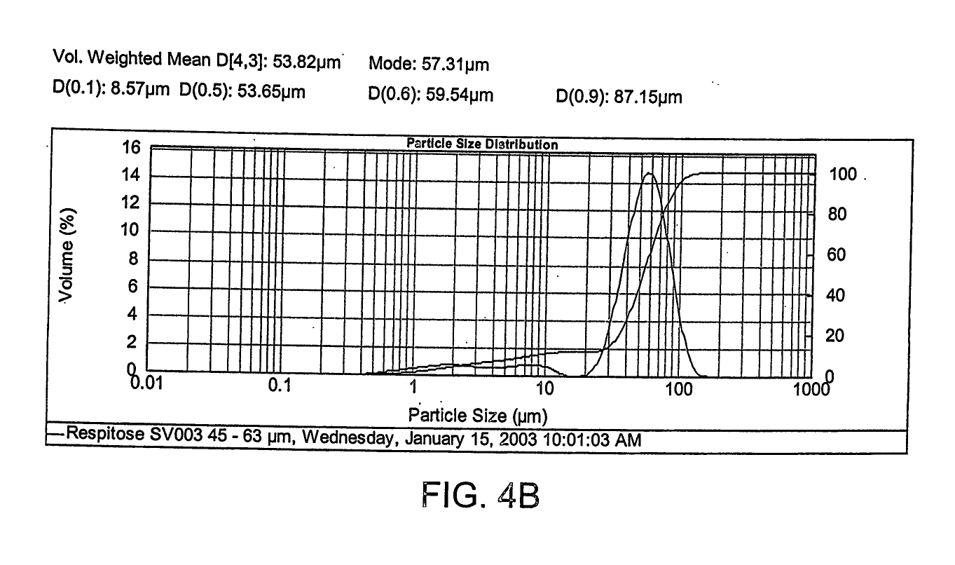

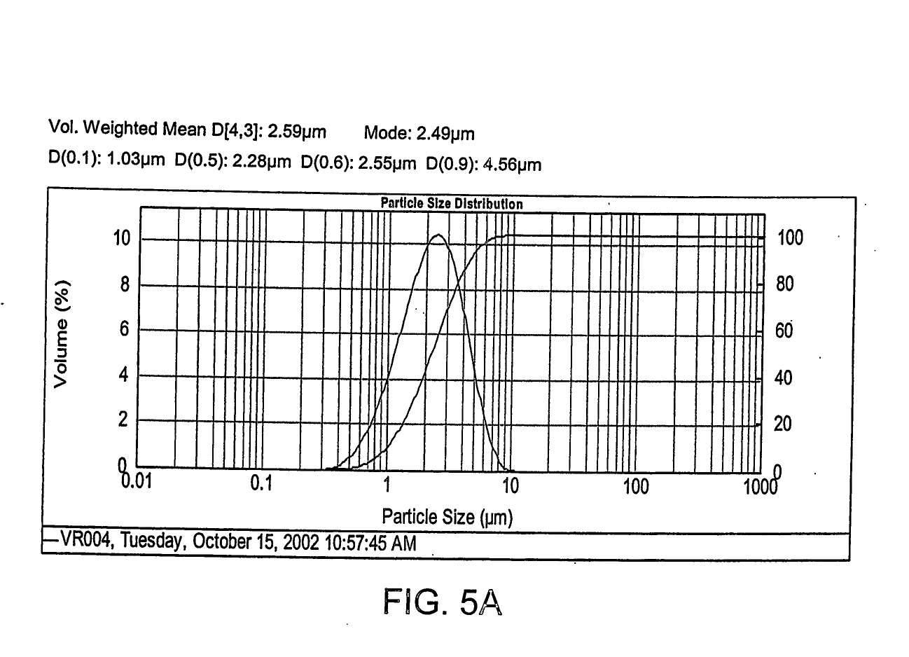

A sieved fraction of Respitose SV003 (DMV International Pharma, The Netherlands) lactose is manufactured by passing bulk material through a 63 μm sieve. This material is then sieved through a 45 μm screen and the retained material is collected. FIGS. 4A and 4B show the results of a particle size analysis of two batches of the lactose performed with a Mastersizer 2000, manufactured by Malvern Instruments, Ltd. (Malvern, UK).

As shown, the lactose had a volume weighted mean of from about 50 to about 55 μm, a d₁₀ of from about 4 to about 10 μm, a d₅₀ of from about 50 to about 55 {m, and a d₉₀ of from about 85 to about 95 μm wherein d₁₀ d₅₀ d₉₀ refer to the diameter of 10%, 50%, and 90% of the analysed lactose.

### Example 2

### Preparation of Apomorphine-Lactose Formulation

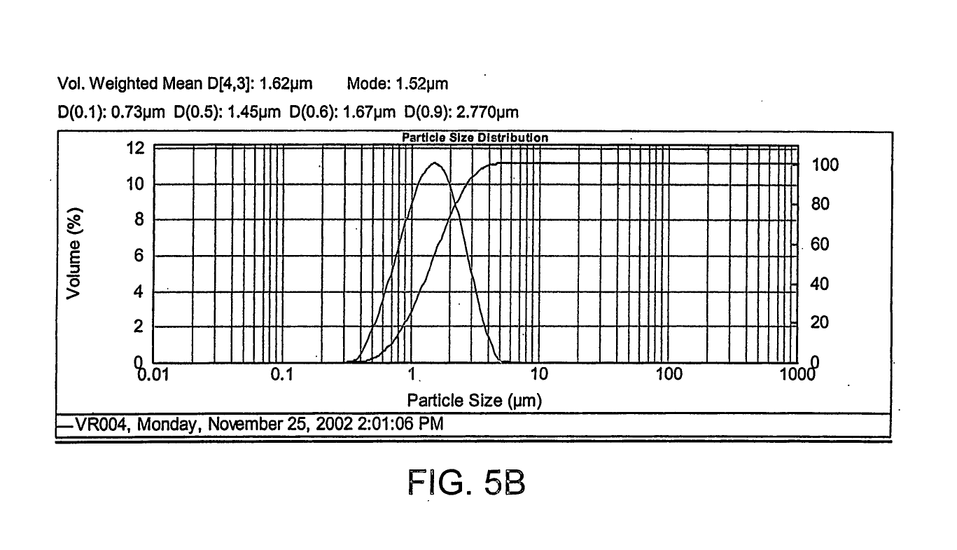

Apomorphine hydrochloride was obtained from Macfarlan Smith Ltd, and was micronised according to the following product specification: ≧99.9% by mass<10 μm, based upon a laser diffraction analysis. Actual typical results of the laser fraction analysis were as follows: d₁₀<1 μm, d₅₀: 1-3 μm; d₉₀<6 μm, wherein d₁₀ d₅₀ d₉₀ refer to the diameter of 10%, 50%, and 90% of the analysed apomorphine hydrochloride. The apomorphine hydrochloride was micronised with nitrogen, (rather than the commonly employed air) to prevent oxidative degradation. FIGS. 5A and 5B show the results of a particle size analysis of two batches of the micronised apomorphine hydrochloride performed with the Mastersizer 2000, manufactured by Malvern Instruments, Ltd. (Malvern, UK).

### Example 2(a)

### Preparation of 200 Microgram Formulation

70 grams of the lactose of Example 1 were placed into a metal mixing vessel of a suitable mixer. 10 grams of the micronised apomorphine hydrochloride were then added. An additional 70 grams of the lactose of Example 1 were then added to the mixing vessel, and the resultant mixture was tumbled for 15 minutes. The resultant blend was then passed through a 150 μm screen. The screened blend (i.e. the portion of the blend that passed through the screen) was then reblended for 15 minutes.

The particle size distribution of the apomorphine-lactose powder, as determined by an Andersen Cascade Impactor (U.S.P. 26, Chapter 601, Apparatus 3 (2003)), showed that the drug particles were well dispersed. In particular, the particle size distribution for a 200 μg dose was as follows:

| | | |
|---|---|---|
| Fine particle dose (<5 μm) | 117 | μg |
| Ultrafine particle dose (<2.5 μm) | 80 | μg |
| MMAD (Mass Median Aerodynamic Diameter) | 1.94 | μm |

### Example 2(b)

### Preparation of 100 Microgram Formulation

72.5 grams of the lactose of Example 1 were placed into a metal mixing vessel of a suitable mixer. 5 grams of the micronised apomorphine hydrochloride were then added. An additional 72.5 grams of the lactose of Example 1 were then added to the mixing vessel, and the resultant mixture was tumbled for 15 minutes. The resultant blend was then passed through a 150 μm screen. The screened blend (i.e. the portion of the blend that passed through the screen) was then reblended for 15 minutes.

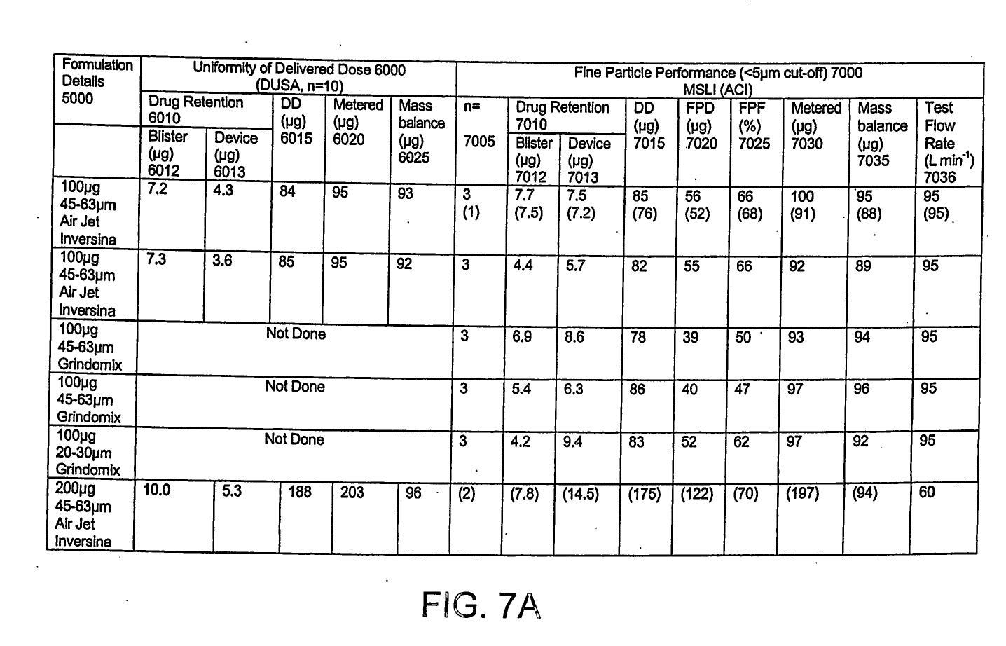

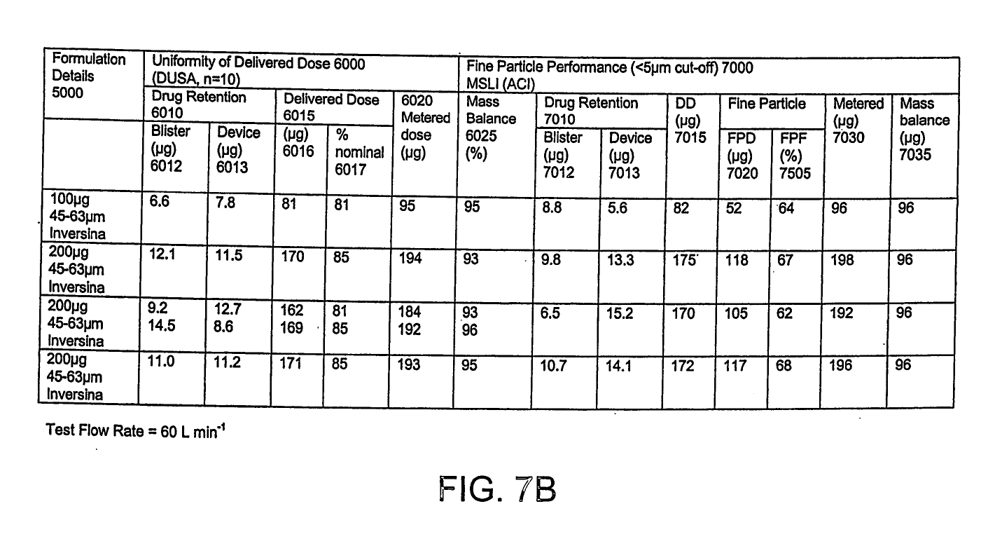

As described below, with reference to FIGS. 7A and 7B, in certain batches of Examples 2(a) and 2(b), the mixer used was an Inversina Variable Speed Tumbler Mixer, which is a low shear mixer distributed by Christison Scientific Equipment Ltd of Gateshead, UK. In other batches, the mixer used was a Retsch Grindomix mixer is a higher shear mixer which is also distributed by Christison Scientific Equipment Ltd. Disaggregation was shown to be sensitive to the intensity of the mixing process but a consistent fine particle fraction (about 60%) was obtained using a low shear mixer equipped with a metal vessel such as the Inversina mixer referenced above.

### Example 3

### Incorporation of Formulation into Blisters

The formulations of Examples 2(a) and 2(b) were each incorporated into blisters in the following manner. Three milligrams of the apomorphine-lactose formulation were placed in each blister. The base of each blister is a cold-formed aluminium blister, formed from a laminate of oriented polyamide (exterior), 45 μm of aluminium (centre), and PVC (interior). The lid of the blister is made of a hard-rolled 30 μm lidding foil, having a heat seal lacquer. After the formulation is loaded into the interior of the blisters, the blisters are sealed by placing the lid over the blister base, and heat sealing the lid to the base via the heat seal lacquer.

During initial development the aluminium/PVC blisters as described above were used. During the course of the study (not for technology reasons) we also tested aluminium/polyethylene (PE) blisters, expecting no difference in performance. However the results shown below in the table below demonstrate that the PE blister material appears to lead to considerably worse performance. There is also evidence that the apomorphine hydrochloride chemically degrades in the presence of the polyethylene.

TABLE 1
Differences of mean drug retention in PE and PVC blisters

| | Mean drug retention | Mean drug retention |
|---|---|---|
| | PE blisters | PVC blisters |
| DUSA | 43.6 μg | 12.9 μg |
| Initial stability | 44.7 μg | 13.9 μg |

Initial stability data using the PE blisters also show an increase in some of the related substance peaks compared to the initial peaks after 1 month this suggests that degradation of the formulated product takes place in the presence of PE. Therefore, the PVC foil blister system is preferred for use with apomorphine hydrochloride. Polypropylene is also an acceptable alternative.

### Example 4

### Stability Data

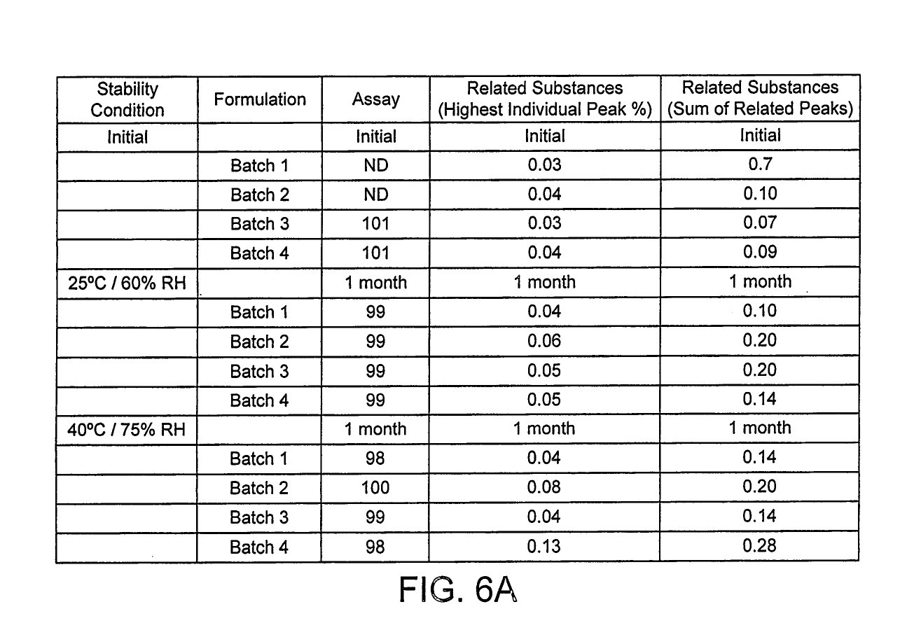

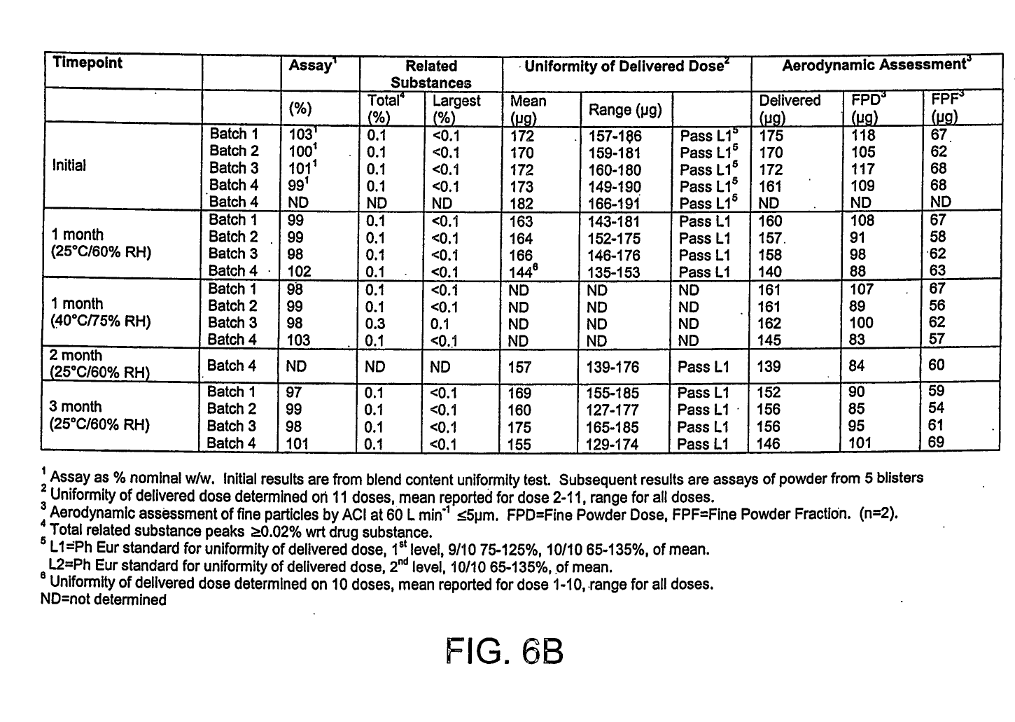

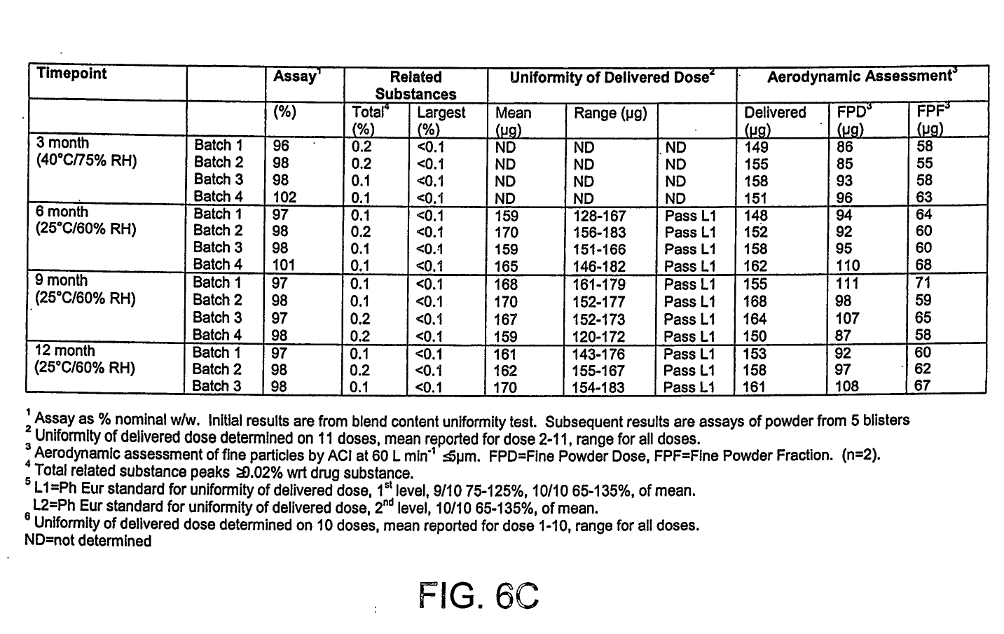

The above referenced blisters containing the apomorphine-lactose formulations of Example 2(a), where each formulation comprises 6.67% drug (200 μg), were placed into heat sealed aluminium laminate bags to replicate patient packs. Storage conditions were at 25° C. and 60% relative humidity, and 40° C. and 75% relative humidity (accelerated storage conditions). The stability data was collected over the course of one year with test dates of 1 month and 3 months for both storage conditions, with additional test dates of 6 months, 9 months and 12 months for blisters stored at 25° C. and 60% relative humidity. The results of the stability tests are shown in FIGS. 6A to 6C.

The chemical stability measures the stability of the drug substance. This is necessary because apomorphine hydrochloride has a reputation for being unstable, particularly in the presence of oxygen/air and water.

To test the chemical stability, the formulation was removed from the laminate bags and the blisters and was tested using High Performance Liquid Chromatography (HPLC). The assay value is the percent of the expected apomorphine content of the formulation, the relative substance (Rel Subs) is the total related substance peaks as a percentage of the total peak area in the chromatogram. As one of ordinary skill in the art will appreciate, these values (shown in FIG. 6A) are well within the acceptable parameters of 0.1% for Rel Subs.

The physical stability was also measured over the same time frame. This is the “performance” aspect of the stability programme, investigating whether the amount of drug delivered to the deep lung will differ over time. The results are set out in FIGS. 6B and 6C.

The uniformity of delivered dose was determined using the Aspirair™ device on 11 DUSAs, where the first shot was not reported, in accordance with standard practice. This means that the uniformity of the delivered dose is calculated on shots 2-11 to give the required n=10. The formulation was 20% drug-blend (made according to the standard example), filled at 3 mg, giving a nominal dose of 600 μg.

The aerodynamic assessment of fine particles was determined using an Andersen Cascade Impactor (ACI) where FPD=Fine Particle Dose of ≦5 μm and FPF=Fine Particle Fraction of ≦5 μm. The flow rate of both the uniformity and the aerodynamic assessment was 60 l/min.

TABLE 4
Machine-filled blisters

Delivered Dose

| | | Mean for Doses 2-11 |
|---|---|---|
| Test No. | Individual | (n = 10) |
| (1 | ND | Mean = 503 |
| 2 | 489 | SD = 14 |
| 3 | 495 | % RSD = 2.7 |
| 4 | 508 | Mean as % of nominal = 89 |
| 5 | 514 | Mass balance (%) = 98 |
| 6 | 532 | |
| 7 | 488 | |
| 8 | 506 | |
| 9 | 509 | |
| 10 | 493 | |
| 11 | 497 | |

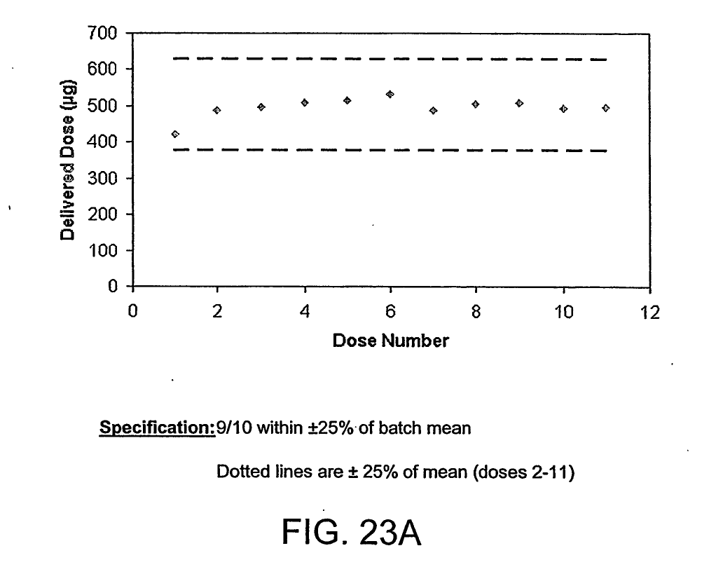

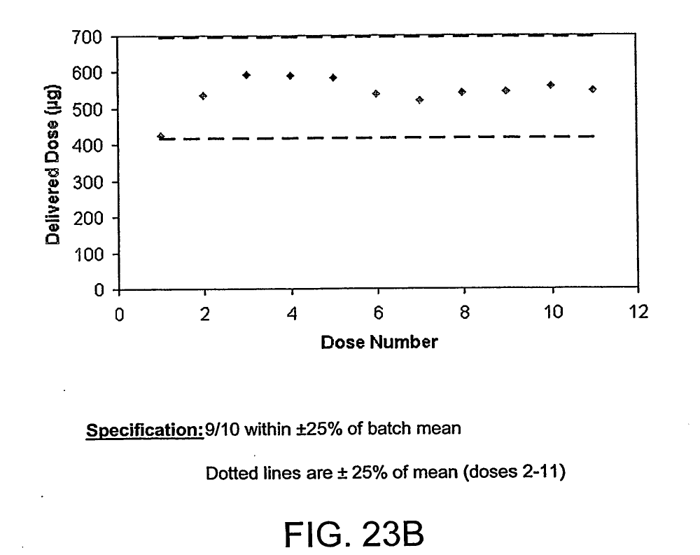

A graph showing the delivered dose (μg) for each of the 10 measured doses is shown in FIG. 23A.

TABLE 5
Hand-filled blisters

Delivered Dose

| | | Mean for Doses 2-11 |
|---|---|---|
| Test No. | Individual | (n = 10) |
| (1 | ND) | Mean = 556 |
| 2 | 536 | SD = 25 |
| 3 | 593 | % RSD = 4.5 |
| 4 | 591 | Mean as % of nominal = 93 |
| 5 | 584 | Mass Balance (%) = 92 |
| 6 | 539 | |
| 7 | 521 | |
| 8 | 543 | |
| 9 | 545 | |
| 10 | 560 | |
| 11 | 548 | |

A graph showing the delivered dose (μg) for each of the 10 measured doses is shown in FIG. 23B.

### Example 5

### Inhalation Testing

Blisters containing the apomorphine-lactose formulations were subjected to testing using an Aspirair prototype inhaler.

In order to obtain the inhalation data described below, the inhaler device was used in conjunction with three instruments, a Multi-Stage Liquid Impinger (MSLI) (U.S.P. 26, Chapter 601, Apparatus 4 (2003), an Anderson Cascade Impactor (ACI) (U.S.P. 26, Chapter 601, Apparatus 3 (2003), and a Dosage Unit Sampling Apparatus (DUSA) (U.S.P. 26, Chapter 601, Apparatus B (2003). Each of these devices has an input for receiving the mouthpiece of the inhaler.

The DUSA is used to measure the total amount of drug which leaves the inhaler. With data from this device, the metered and delivered dose is obtained. The delivered dose is defined as the amount of drug that leaves the inhaler. This includes the amount of drug in the throat of the DUSA device, in the measuring section of the DUSA device and the subsequent filters of the DUSA device. It does not include drug left in the blister or other areas of the inhaler, and does not account for drug “lost” in the measuring process of the DUSA device. The metered dose includes all of the drug which leaves the blister.

The MSLI is a device for estimating deep lung delivery of a dry powder formulation. The MSLI includes a five stage cascade impactor which can be used for determining the particle size (aerodynamic size distribution) of Dry Powder Inhalers (DPIs) in accordance with USP 26, Chapter 601, Apparatus 4 (2003) and in accordance with the European Pharmacopoeia, Method 5.2.9.18, Apparatus C, Supplement 2000.

The ACI is another device for estimating deep lung delivery of a dry powder formulation. The ACI is multi-stage cascade impactor which can be used for determining the particle size (aerodynamic size distribution) of dry powder inhalers (DPI) in accordance with USP 26, Chapter 601, Apparatus 3 (2003).

As described below, the MSLI and the ACI testing devices can be used to determine, inter alia, the fine particle dose (FPD), i.e. the amount of drug, e.g., in micrograms, that is measured in the sections of the testing device which correlates with deep lung delivery and the fine particle fraction (FPF), i.e. the percentage of the metered dose which is measured in the sections of the testing device which correlates with deep lung delivery.

FIGS. 7A and 7B illustrate the results of tests performed on the apomorphine-lactose formulation of Example 2. The FPD, FPF and MMAD values were generated from the MSLI and ACI data using the Copley Inhaler Data Analysis Software (CITDAS) V1.12. In FIG. 7A, data is shown for six formulations, which are identified in column 5000. FIG. 7B provides data for an additional four formulations. In each Figure, the test data for the formulations is divided into two types: data relating to uniformity of the delivered dose for the formulations (column 6000) and data relating to fine particle size performance of the formulations (column 7000).

Referring to FIG. 7A, the first five formulations listed in column 5000 include 3 mg of the 100 microgram formulation of Example 2(b). The sixth formulation listed includes 3 mg of the 200 microgram formulation of Example 2(a). The first, second, and sixth formulation listings in 5000 contain the notation “Inversina” to indicate that the mixer used in Example 2 was the Inversina Mixer, and the third, fourth, and fifth formulation listing contain the notation “Grindomix” to indicate that the mixer used in Example 2 was the Grindomix Mixer. The second and fourth formulations listed also contain the notation “Air Jet” to indicate that for these formulations the lactose in Example 1 was sieved with an Air Jet Sieve which applies a vacuum to the screen sieve apparatus, rather than a conventional screen sieve (which was used for the first third, fifth, and sixth formulations listed). The fifth formulation listed also contains the notation “20-30 μm Extra Fine” to indicate the approximate particle size range for this material.

In section 6000 of FIG. 7A, the DUSA apparatus described above is used to provide data for the formulations regarding the drug retention in the blister (6012), the drug retention in the inhaler (6013), the delivered dose (6015), the metered dose (6020), and the mass balance percentage (6025). The notation n=10 indicates that the inhaler and DUSA apparatus was fired 10 times for each of the three formulations for which DUSA data is listed. The data listed in section 6000 is an average of the 10 firings.

In section 7000 of FIG. 7B, the fine particle performance is measured with two different devices, the MSLI and the ACI. Data for the ACI, where available, is indicated in parenthesis ( ). In any event, the data provided in section 7000 is for particles having a particle size diameter of less than 5 μm (referred to in this discussion as “fine particles”). As such, column 7012 provides the fine particle drug retention in the blister, column 7013 provides the fine particle drug retention in the inhaler, column 7015 provides the amount of fine particles in the delivered dose, column 7020 provides the FPD for the formulation, column 7025 provides the FPF for the formulation, column 7015 provides the amount of fine particles in the metered dose, column 7035 provides the mass balance percentage for the formulations in the MSLI (ACI) tests, and column 7036 provides the test flow rate for the formulations. Column 7005 indicates that the number of times the inhaler and MSLI (or ACI) apparatus were fired, and the data listed is an average of the “n” firings.

FIG. 7B is similar to FIG. 7A, with similar items bearing identical reference numbers. The first formulation listed in column 5000 include 3 mg of the 100 microgram formulation of Example 2(b), the remaining four formulations include 3 mg of the 200 microgram formulation of Example 2(a), and all of the formulations were made with the Inversina Mixer, and were prepared with lactose prepared using 45 and 63 μm screens. The DUSA data in column 6000 was obtained in the same manner as in FIG. 7A, except that n=11. All of the fine particle performance data in section 7000 was obtained using the ACI apparatus with n=2, and a flow rate of 60 L min⁻¹.

As illustrated in FIGS. 7A and 7B, when the formulations were mixed using the low shear Inversina mixer, the fine particle fraction (FPF) ranged from a low of 62% to a high of 70%, and the percent delivered dose ranged from a low of 81% to a high of 94%. The formulations made with the higher shear Grindomix mixer exhibited a fine particle fraction of from 47% to 50% for formulations including the 45-63 μm lactose. The formulation made with the high shear Grindomix mixer and with lactose having a particle size between 20 and 30 μm exhibited an increased fine particle fraction of 62%.

### Example 6

### Preparation of 400 Microgram Formulation in 3 mg Blister

A 400 microgram formulation can be manufactured in the manner set forth above with regard to Example 2, with the components provided in the following amounts:

| Composition | Amount (μg) | Percent |
|---|---|---|
| Apomorphine HCl | 400 | 13.33 |
| Lactose | 2600 | 86.66 |
| Total | 3000 | 100 |

### Example 7

### Preparation of 600 Microgram Formulation in 3 mg Blister

A 600 microgram formulation can be manufactured in the manner set forth above with regard to Example 2, with the components provided in the following amounts:

| Composition | Amount (μg) | Percent |
|---|---|---|
| Apomorphine HCl | 600 | 20 |
| Lactose | 2400 | 80 |
| Total | 3000 | 100 |

Although the above referenced examples utilize a blister “fill weight” of 3 mg, it should be appreciated that larger or smaller fill weights may also be used. For example, in Examples 8-12 below, fill weights of 1 mg or 2 mg are provided. Although a variety of techniques for filling blisters to such fill weights may be used, it is known that commercial production of blisters with between 1 mg and 5 mg fill weights has been achieved with a Harro-Hoefliger Omnidose Drum Filler.

### Example 8

### Preparation of 800 Microgram Formulation in 2 mg Blister

An 800 microgram formulation can be manufactured in the manner set forth above with regard to Example 2, with the components provided in the following amounts:

| Composition | Amount (μg) | Percent |
|---|---|---|
| Apomorphine HCl | 800 | 26.66 |
| Lactose | 1200 | 73.33 |
| Total | 2000 | 100 |

### Example 9

### Preparation of 200 Microgram Formulation with Magnesium Stearate in 1 mg Blister

A 200 microgram formulation can be prepared including magnesium stearate with the components provided in the following amounts:

| Composition | Amount (μg) | Percent |
|---|---|---|
| Apomorphine HCl | 200 | 20.00 |
| Lactose | 797.5 | 79.75 |
| Magnesium stearate | 2.5 | 0.25 |
| Total | 1000 | 100 |

This formulation can be prepared in the manner set forth above with regard to Example 2, except that magnesium stearate is added to the mixture along with the apomorphine hydrochloride.

### Example 10

### Preparation of 400 Microgram Formulation with Leucine in 2 mg Blister

A 400 microgram formulation can be prepared with leucine with the components provided in the following amounts:

| Composition | Amount (μg) | Percent |
|---|---|---|
| Apomorphine HCl | 400 | 20 |
| Lactose | 1560 | 78 |
| Micronised leucine | 40 | 2 |
| Total | 2000 | 100 |

This formulation can be prepared in the manner set forth above with regard to Example 2, except that micronised leucine is added to the mixture along with the apomorphine hydrochloride.

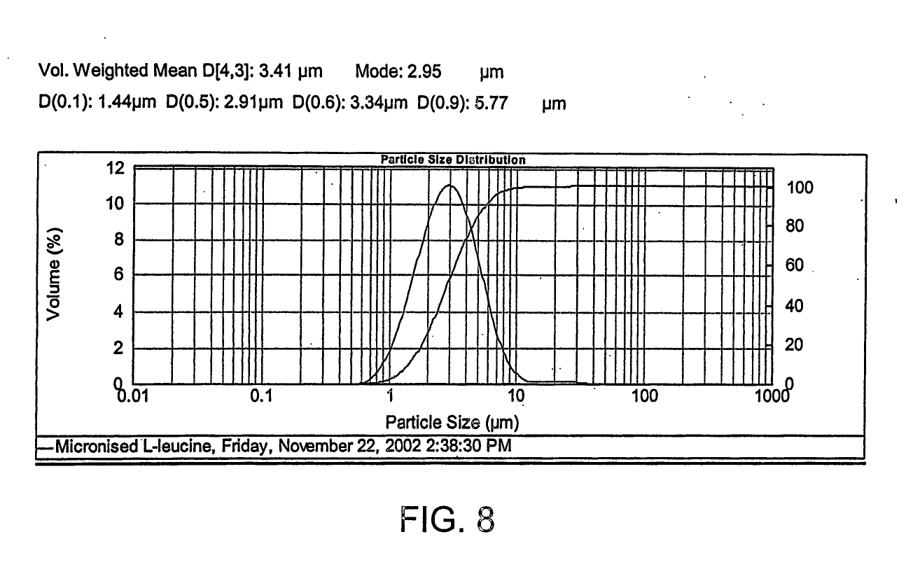

FIG. 8 shows the results of a particle size analysis of a preferred micronised leucine performed with the Mastersizer 2000, manufactured by Malvern Instruments, Ltd. (Malvern, UK). As illustrated, the exemplified micronised leucine has a volume weighted mean particle diameter of 3.4 μm, with ⁹⁰% of the particles having a volume weighted mean particle diameter of less than 6 μm.

### Example 11

### Preparation of 200 Microgram Formulation in 2 mg Blister

A 200 microgram formulation can be manufactured in the manner set forth above with regard to Example 2, with the components provided in the following amounts:

| Composition | Amount (μg) | Percent |
|---|---|---|
| Apomorphine HCl | 200 | 10 |
| Lactose | 1800 | 90 |
| Total | 2000 | 100 |

### Example 12

### Preparation of 200 Microgram Formulation in 1 mg Blister

A 200 microgram formulation can be manufactured in the manner set forth above with regard to Example 2, with the components provided in the following amounts:

| Composition | Amount (μg) | Percent |
|---|---|---|
| Apomorphine HCl | 200 | 20 |
| Lactose | 800 | 80 |
| Total | 1000 | 100 |

### Example 13

### Preparation of 400 Microgram Formulation in 2 mg Blister

A 400 microgram formulation can be manufactured in the manner set forth above with regard to Example 2, with the components provided in the following amounts:

| Composition | Amount (μg) | Percent |
|---|---|---|
| Apomorphine HCl | 400 | 20 |
| Lactose | 1600 | 80 |
| Total | 2000 | 100 |

### Example 14

### In Vivo Clinical Data from Patients Treated with Apomorphine Via DPI Inhalation

In this study, 35 volunteer patients were given 4 random doses of placebo, 200 μg of apomorphine hydrochloride, 400 μg of apomorphine hydrochloride or 800 μg of apomorphine hydrochloride. The doses were administered using an Aspirair prototype device either with the blister of Example 3 (200 μg of apomorphine hydrochloride in a 3 mg blister) or in a placebo blister (lactose only).

During each treatment, a patient was administered the given dose and was left alone to watch an hour of visual sexual stimulation (VSS). At 50-55 minutes after administration, the patients were warned that the study would end at 60 minutes. After 60 minutes, the patient's were asked to rate the quality and duration of their response to VSS. In this regard, the quality of response is defined as one of four grades: 0: no effect; 1: some tumescence, no rigidity; 2: some tumescence, some rigidity, but not suitable for penetration; 3: rigidity and tumescence that would enable penetration but is not complete erection; 4: complete erection.

This study was conducted in a double blind fashion, where both the healthcare professional administering the treatment and the patient were not informed as to the actual dose being administered. The patients who participated in this study were randomised. During each treatment, each of the 35 patients received 4 blisters regardless of the dose i.e., a patient receiving a 400 μg HCl dose would receive 2 (two) of the apomorphine HCl blisters and 2 (two) of the placebo blisters and a patient receiving only placebo took 4 (four) of the placebo blisters.

The study showed that the groups treated with 400 μg and 800 μg of apomorphine HCl experienced the quickest onset of effect, longest duration and most complete erections as compared to the groups treated with either placebo or 200 μg apomorphine HCl dose. For example, the group treated with 800 μg apomorphine HCl exhibited a median onset of effect in about 8 or less minutes after administration of apomorphine HCl as compared to about 11 or less minutes for the 200 μg apomorphine HCl group, based upon grade 3 and 4 responders. Grade 3 or 4 responses were achieved as quickly as 4 minutes for the 400 and 800 μg groups. It is believed that if this treatment were to be repeated with single dosing as opposed to 4 doses at a time (i.e. one 800 μg blister dose), the response to treatment would exhibit an even faster onset, thereby, providing even more effective treatment.

In the study, patients treated with placebo (4 blisters, each consisting of placebo) showed a 31.4% average response rate. The 200 μg group (4 blisters, 1 containing 200 μg apomorphine HCl and the remaining 3 blisters each containing placebo) showed a 22.9% average response rate, the 400 μg group (4 blisters, 2 containing 200 μg apomorphine HCl and the remaining 2 containing placebo) showed a 48.5% average response rate, and the 800 μg group (4 blisters, each containing 200 μg apomorphine HCl) showed a 58.8% average response rate. As the patients treated with 400 μg and 800 μg displayed significantly higher response rates as compared to those patients treated with either placebo or 200 μg, the 400 μg and 800 μg doses are considered to be effective (see Table 6 below).

TABLE 6
Summary of response rate (ITT population)

| Dose | Evaluated | Responding | Rate (%) | CI Limit¹ | Effective? |
|---|---|---|---|---|---|
| Placebo | 35 | 11 | 31.4% | 18.7% | No |
| 200 μg | 35 | 8 | 22.9% | 11.9% | No |
| 400 μg | 33 | 16 | 48.5% | 33.3% | Yes |
| 800 μg | 34 | 20 | 58.8% | 43.3% | Yes |

¹The confidence interval (CI) is a one sided 95% CI. It extends from the limit shown to 100%.

The primary measure of efficacy, as defined in the protocol, was the proportion of subjects reporting a grade 3 or 4 erection, using general criteria defined in the International Index of Erectile Function (IIEF). Grade 3 and 4 erections are regarded as “sufficient for successful intercourse”. Using these criteria, the 400 μg and 800 μg doses of apomorphine HCL were deemed effective.

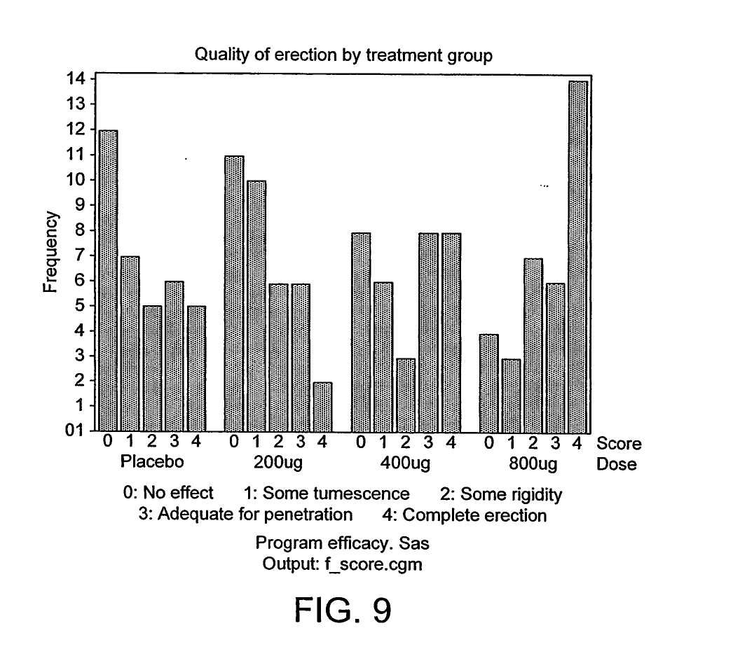

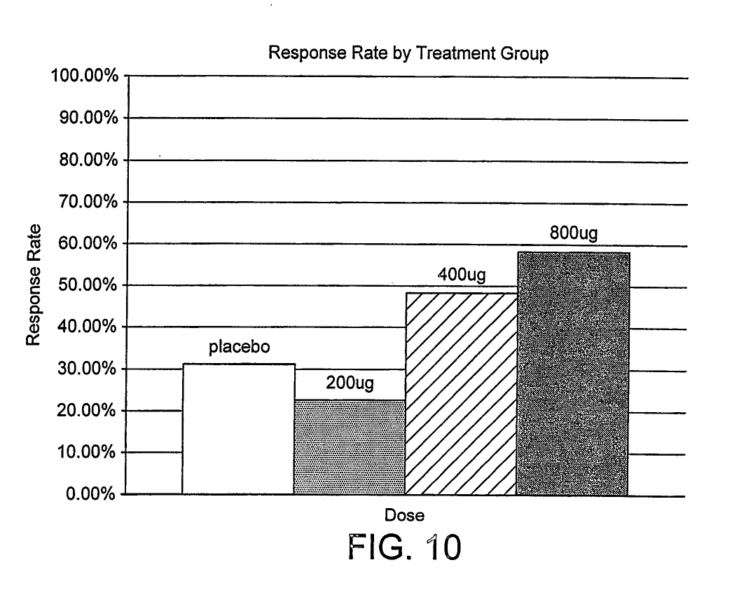

As illustrated in FIGS. 9 and 10, a clear dose response relationship was noted amongst the active dose groups, both in the proportion of “sufficient” erections, the proportion of grade 4 or “full” erections and response rate. For example, the group treated with 800 μg of apomorphine HCl showed the greatest number of grade 4 erections, highest response rate and quickest onset of effect in comparison to the groups treated with placebo, 200 μg and 400 μg of apomorphine HCl.

With respect to efficacy, Table 7 below illustrates that the 200 μg apomorphine HCl dose group exhibited a median onset of effect of 11 minutes after administration (with a standard of deviation of 4.2), and the placebo group exhibited a median onset of effect of 10 minutes after administration (with a standard of deviation of 7.8). In contrast, the 400 μg and 800 μg apomorphine HCl dose groups exhibited the quickest median onset of effect (8 (SD 7.5) and 8 (SD 5.0) respectively). The 400 μg and 800 μg apomorphine HCl dose groups also exhibited the most complete erections and highest response rate percentages as compared to the groups treated with either 200 μg apomorphine HCl or placebo.

TABLE 7
Summary of efficacy (ITT population)

| | Quality | Treatment | | | |
|---|---|---|---|---|---|
| Quality | Grade | Placebo | 200 μg | 400 μg | 800 μg |
| No effect | 0 | 12 | 11 | 8 | 4 |
| Some tumescence, no | 1 | 7 | 10 | 6 | 3 |
| rigidity | | | | | |
| Some tumescence and | 2 | 5 | 6 | 3 | 7 |
| rigidity | | | | | |
| Partial erection | 3 | 6 | 6 | 8 | 6 |
| Full erection | 4 | 5 | 2 | 8 | 14 |
| Onset (min post dose) | N | 11 | 8 | 16 | 19 |
| | Mean | 13 | 13 | 11 | 10 |
| | SD | 7.8 | 4.2 | 7.5 | 5.0 |
| | Min | 4 | 8 | 3 | 3 |
| | Max | 27 | 20 | 28 | 17 |
| | Median | 10 | 11 | 8 | 8 |
| Duration (min) | N | 11 | 8 | 16 | 19 |
| | Mean | 29 | 33.3 | 31.1 | 31.2 |
| | SD | 18.0 | 7.4 | 8.4 | 16.6 |
| | Min | 6 | 24 | 4 | 6 |
| | Max | 52 | 47 | 54 | 54 |
| | Median | 30.0 | 31.5 | 38 | 36 |

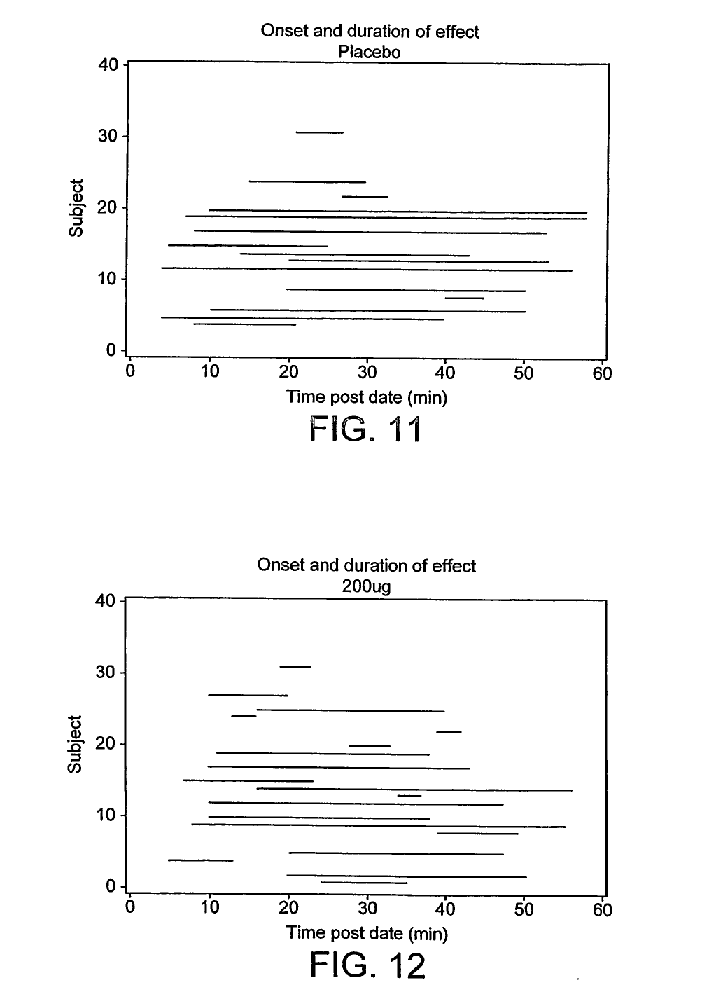

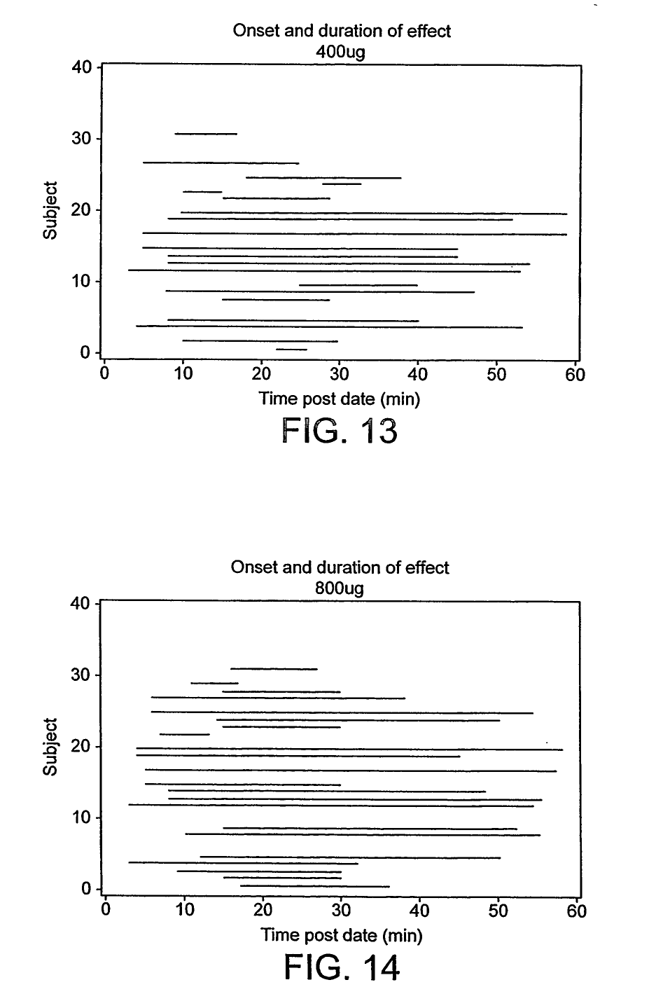

A more detailed illustration of the onset and duration of effect for each individual group is provided in FIGS. 11 through 14. FIG. 11 shows the onset and duration of effect for the patients who were treated with placebo. FIG. 12 shows the onset and duration of effect for the patients treated with 200 μg apomorphine HCl. FIG. 13 shows the onset and duration of effect for the patients treated with 400 μg apomorphine HCl and FIG. 14 shows the onset and duration of effect for the patients treated with 800 μg apomorphine HCl. For example, referring to FIG. 14, it is apparent that one patient in the 800 μg apomorphine HCl group experienced the onset of an erection at about 4 minutes after administration. Referring to FIG. 13, for example, it is apparent that a patient in the 400 μg apomorphine HCl group experienced the onset of an erection at about 3 minutes after administration. In contrast, FIG. 12 shows that one patient in the 200 μg group experienced the onset an erection at about 40 minutes after administration. Overall, these Figures illustrate that the groups that received 400 μg and 800 μg doses of apomorphine HCl experienced faster onset of erections. It should be appreciated that the testing period lasted 60 minutes, and the patients were reminded at 50-55 minutes that the test would end at 60 minutes.

Adverse events were monitored during each dosing period. The proportion of patients experiencing one or more adverse events was similar in all four treatment groups. No serious adverse events were observed and no adverse event led to the premature discontinuation of any subject. All adverse events were mild or moderate in severity and occurred in a small percentage of the groups treated. Table 8 is a summary of all adverse events. Table 9 is a summary of all treatment related adverse events, and Table 10 breaks treatment-related adverse events down by body system.

Referring to Table 8, only 6% of the 800 μg apomorphine HCl group experienced adverse events, which is the same percentage of those who experienced adverse events in both the placebo and 200 μg apomorphine HCl group.

TABLE 8
Summary of all adverse events (AE) (Safety population)

| | Placebo | | 200 μg | | 400 μg | | 800 μg | |
|---|---|---|---|---|---|---|---|---|
| | N | % | N | % | N | % | N | % |
| Subjects treated | 35 | | 35 | | 35 | | 35 | |
| With AE | 4 | 11% | 3 | 9% | 3 | 9% | 2 | 6% |
| With severe AEs | 0 | | 0 | | 0 | | 0 | |
| With serious AEs | 0 | | 0 | | 0 | | 0 | |
| Discontinued due | 0 | | 0 | | 0 | | 0 | |
| to AE | | | | | | | | |

TABLE 9
Summary of treatment-related adverse events (AE) (Safety population)

| | Placebo | | 200 μg | | 400 μg | | 800 μg | |
|---|---|---|---|---|---|---|---|---|
| | N | % | N | % | N | % | N | % |
| Subjects treated | 35 | | 35 | | 35 | | 35 | |
| With AE | 2 | 6% | 2 | 6% | 3 | 9% | 2 | 6% |
| With severe AEs | 0 | | 0 | | 0 | | 0 | |
| With serious AEs | 0 | | 0 | | 0 | | 0 | |
| Discontinued due | 0 | | 0 | | 0 | | 0 | |
| to AE | | | | | | | | |

TABLE 10
Treatment-related adverse events by body system (safety population)

| Body system/ | Placebo | | 200 μg | | 400 μg | | 800 μg | |
|---|---|---|---|---|---|---|---|---|
| Preferred term | N | % | N | % | N | % | N | % |
| Subjects treated | 35 | | 35 | | 35 | | 35 | |
| Gastrointestinal disorders | 1 | 3% | 0 | | 0 | | 1 | 3% |
| Nausea | 0 | | 0 | | 0 | | 1 | 3% |
| Vomiting NOS | 1 | 3% | 0 | | 0 | | 0 | |
| Nervous system disorders | 1 | 3% | 1 | 3% | 0 | | 2 | 6% |
| Dizziness | 0 | | 1 | 3% | 0 | | 2 | 6% |
| Headache | 1 | 3% | 0 | | 0 | | 0 | |
| Respiratory, thoracic & | 2 | 6% | 1 | 3% | 3 | 9% | 0 | |
| mediastinal disorders | | | | | | | | |
| Cough | 1 | 3% | 1 | 3% | 0 | | 0 | |
| Dry throat | 1 | 3% | 0 | | 1 | 3% | 0 | |
| Nasal congestion | 0 | | 1 | 3% | 0 | | 0 | |
| Pharyngolaryngeal pain | 0 | | 0 | | 2 | 6% | 0 | |
| Sneezing | 0 | | 1 | 3% | 0 | | 0 | |

For each patient, blood samples were taken 70 minutes after inhalation. The blood samples were analysed, and the blood levels for 400 and 800 microgram doses of apomorphine for each of the 34 patients that completed the test are set forth in Table 11 in nanograms per millilitre. It should be appreciated from the data discussed in Example 15 below that these blood samples were actually taken long after the plasma concentration peak.

TABLE 11
Blood analysis 70 minutes after dosing

| Patient ID | Apomorphine HCl 800 μg | Apomorphine HCl 400 μg |
|---|---|---|
| Sub 1 | 0.540 | 0.138 |
| Sub 2 | 0.829 | 0.293 |
| Sub 3 | 0.716 | 0.233 |
| Sub 4 | 0.456 | 0.256 |
| Sub 5 | 0.468 | 0.300 |
| Sub 6 | 0.656 | 0.274 |
| Sub 7 | 0.550 | 0.133 |
| Sub 8 | 0.740 | 0.424 |
| Sub 9 | 0.824 | 0.271 |
| Sub 10 | 0.415 | 0.153 |
| Sub 11 | 0.585 | 0.253 |
| Sub 12 | 0.570 | 0.240 |
| Sub 13 | 0.271 | 0.140 |
| Sub 14 | 0.563 | 0.398 |
| Sub 15 | 0.549 | 0.294 |
| Sub 16 | 0.367 | 0.171 |
| Sub 17 | 0.504 | 0.219 |
| Sub 19 | 0.756 | 0.000 |
| Sub 20 | 0.467 | 0.214 |
| Sub 21 | 0.646 | 0.207 |
| Sub 22 | 0.734 | 0.226 |
| Sub 23 | 0.648 | 0.263 |
| Sub 24 | 0.598 | 0.205 |
| Sub 25 | 0.384 | 0.188 |
| Sub 26 | 0.730 | 0.167 |
| Sub 27 | 0.437 | 0.174 |
| Sub 28 | 0.414 | 0.132 |
| Sub 29 | 1.040 | 0.109 |
| Sub 30 | 0.593 | 0.220 |
| Sub 31 | 1.471 | 0.126 |
| Sub 32 | 0.446 | 0.251 |
| Sub 33 | 0.501 | 0.244 |
| Sub 34 | 0.405 | 0.177 |
| Sub 35 | 0.808 | 0.213 |
| Mean | 0.608 | 0.215 |
| Median | 0.567 | 0.217 |

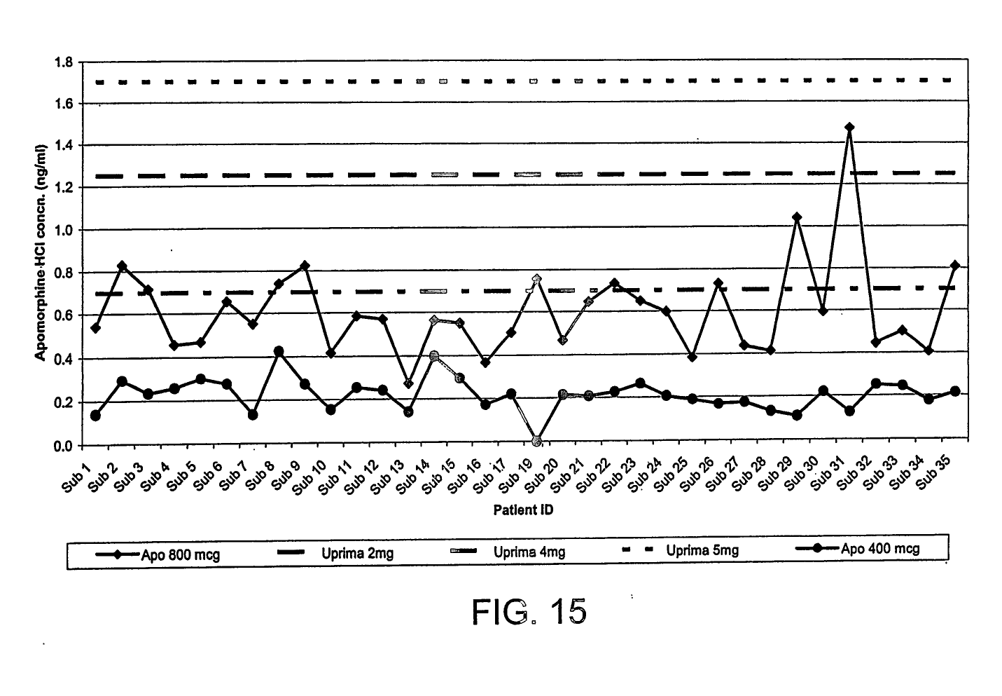

FIG. 15 shows a comparison of the blood levels at 70 minutes after dosing (T₇₀) for each patient for the 400 microgram dose and the 800 microgram dose. Also plotted is the known mean Cₘₐₓ of 2 mg (0.7 ng/ml), 4 mg (1.25 ng/ml), and 5 mg (1.7 ng/ml) of Uprima™ sublingual tablets. In this regard, 4 mg and 5 mg Uprima sublingual tablets are known to have unacceptable side effects. For example, the 4 mg Uprima sublingual tablets were found to have unacceptable clinical safety by the European Agency for the Evaluation of Medicinal Products (see EPAR (European Public Assessment Safety Report) 1945, Uprima, common name apomorphine hydrochloride, “Scientific Discussion”, pp. 25-27 (2002)).

The clinical data described above in connection with Tables 4-6 and the blood level data of Table 11 support the conclusion that the inhaled apomorphine in accordance with the embodiments of the present invention minimizes the risk of side effects.

First, therapeutic (pharmacological) effects are usually dependent upon the value of Cₘₐₓ. However, side effects are often dependent upon the systemic exposure to the drug. Systemic exposure can be measured as the integral of the plasma level from time of administration until it returns to zero (i.e. the area under the curve AUC_{0 to %)}. The measured values of Table 11 demonstrate that plasma levels fall rather rapidly to low values after dosing via inhalation in accordance with the invention. In contrast, absorption is much less rapid and complete by most other routes of administration. For example, EPAR 1945 reports that the elimination half-life for Uprima is 2.7 hours for a 2 mg sublingual dose, 4.2 hours for a 4 mg sublingual dose, 3.9 hours for a 5 mg sublingual dose, and 4.0 hours for a 6 mg sublingual dose. (EPAR 1945, “Scientific Discussion”, p. 12).

A second but equally important beneficial effect of the short half-life associated with the inhaled formulation is that the period in which therapeutic and any side effects is short due to the short half-life of the formulation. Consequently, side effects, if they occur, will be short lived, allowing the patient to resume normal activities such as driving.

### Example 15

### Phase I Study

A phase I, double blind, randomised, placebo controlled study was conducted examining the safety, tolerability and pharmacokinetics of single 600 μg, 900 μg and 1200 μg doses in 16 healthy male volunteers. No evaluation for efficacy was conducted during the clinical study.

Pharmacokinetic plasma sampling was conducted pre-dose and at the following intervals post dose administration: 1 minute, 3 minutes, 5 minutes, 10 minutes, 15 minutes, 30 minutes, 45 minutes, 1 hour, 1.5 hours, 2 hours, 4 hours, 8 hours, 12 hours and 24 hours.

The following pharmacokinetic parameters were derived from the plasma apomorphine concentrations by non-compartmental analysis.

| | |
|---|---|
| Cₘₐₓ | Maximum plasma concentration [ng/ml] |
| tₘₐₓ | Time at which Cₘₐₓ occurs |
| AUC₀₋ₜ | Area under curve [ng/ml * hr] from t = 0 to last |
| | quantifiable concentration |
| AUC_{0-∞} | Area under curve [ng/ml * hr] from t = 0 to infinity |
| t_{1/2} | Terminal elimination half-life |

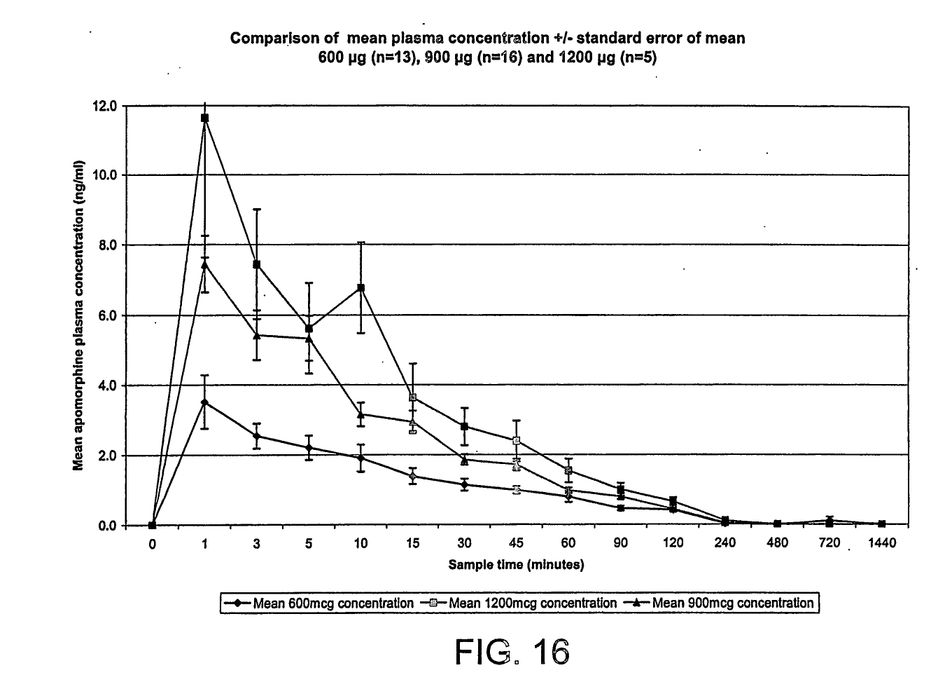

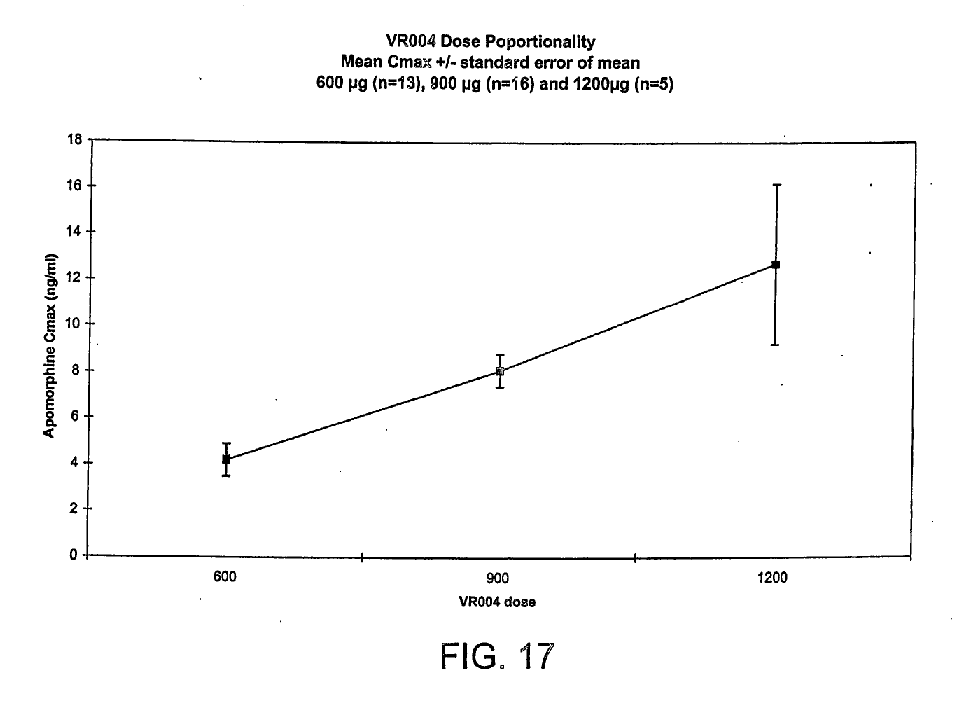

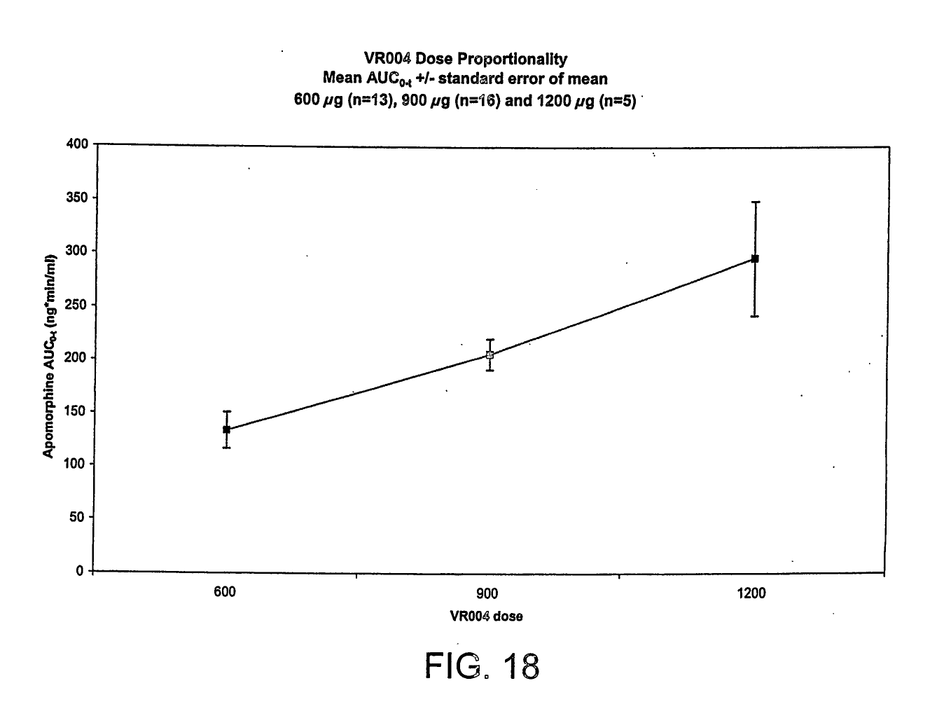

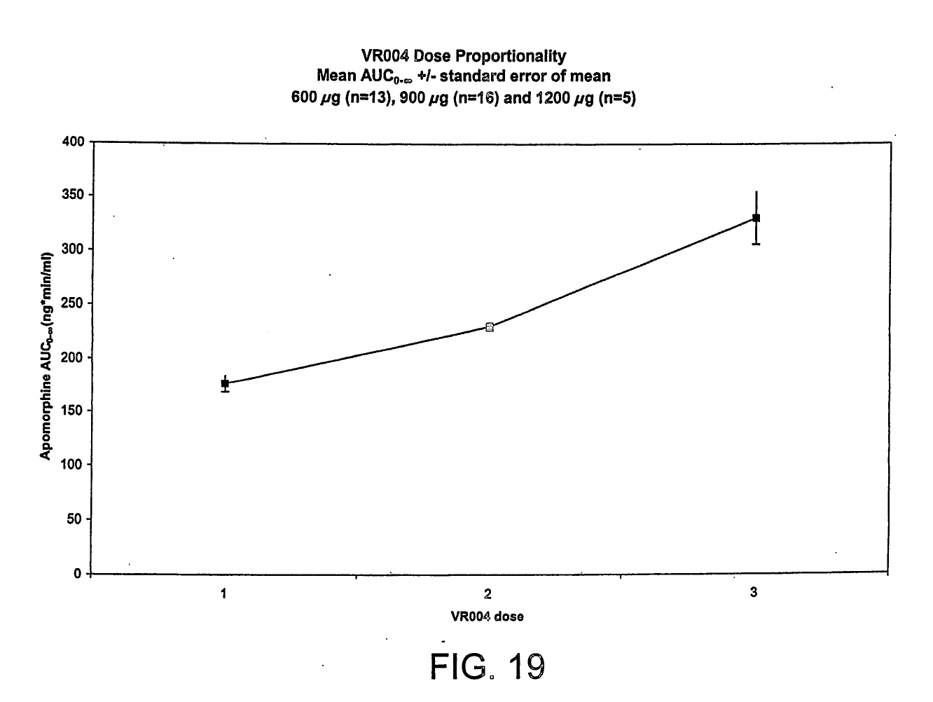

The results are set out in FIGS. 16 to 19 and are summarised in Tables 12 and 13 below. It should be noted that the tₘₐₓ is represented as median values.

TABLE 12
Mean ± standard error of apomorphine plasma pharmacokinetic
parameters

| Dose | | | AUC₀₋ₜ | AUC_{0-∞} | |
|---|---|---|---|---|---|
| Admin- | Cₘₐₓ | tₘₐₓ | (ng * min/ | (ng * min/ | t½ |
| istered | (ng/ml) | (min) | ml) | ml) | (min) |
| 600 μg | 4.2 ± 0.7 | 3 (1-30) | 133.8 ± 17.2 | 176.4 ± 26.9 | 62.9 ± 8.3 |
| 900 μg | 8.1 ± 0.7 | 1 (1-5) | 205.2 ± 14.3 | 230.7 ± 14.0 | 55.4 ± 3.2 |
| 1200 μg | 12.7 ± 3.5 | 1 (1-5) | 295.7 ± 45.7 | 329.9 ± 53.8 | 61.2 ± 7.7 |

TABLE 13
Comparison of inhaled apomorphine and Uprima ® pharmacokinetics

| | Uprima ®¹ | | | | Apomorphine HCl | | |
|---|---|---|---|---|---|---|---|
| Parameter | 2 mg | 4 mg | 5 mg | 6 mg | 600 μg | 900 μg | 1200 μg |
| Cₘₐₓ | 0.7 | 1.3 | 1.7 | 1.9 | 4.2 | 8.1 | 12.7 |
| (ng/ml) | | | | | | | |
| AUC_{0-∞} | 1.2 | 2.4 | 2.9 | 3.6 | 2.2 | 3.4 | 5.5 |
| (ng * h/ml) | | | | | | | |
| t_{1/2} (h) | 2.7 | 4.2 | 3.9 | 4.0 | 1.1 | 0.9 | 1.0 |
| tₘₐₓ | 0.7 h | 0.7 h | 0.7 h | 0.7 h | 3 mins | 1 min | 1 min |

¹European Public Assessment Report, revision 1, 18/12/02, Scientific Discussion

The figures shown in Table 13 indicate that significantly higher Cₘₐₓ values are achieved using the present invention compared to the sublingual Uprima™ tablets. At doses of 600 μg and 900 μg administered by inhalation, no significant side effects were observed. The administration of the 1200 μg dose was associated with high incidence of light-headedness but not the more serious side effects of syncope and vomiting often observed with apomorphine. In contrast, only the 2 mg and 3 mg Uprima tablets are commercially available, as the larger doses cause unacceptable side effect profiles.

Thus, it has surprisingly been found that the administration of apomorphine by pulmonary inhalation according to the present invention achieves much higher blood levels compared to the mode of administration favoured in the prior art, but these high blood levels are not associated with significant side effects.

The following conclusions could be drawn from the study. Rapid systemic absorption with maximum apomorphine plasma concentrations was observed between 1 and 3 minutes after dosing. Dose proportionality was demonstrated for Cₘₐₓ. AUC_{0-∞} and AUC₀₋ₜ. The elimination of drug from the plasma is relatively rapid with a terminal half-life of approximately 60 minutes observed for all study doses. The elimination half-life appears to be dose independent.

It is important to note that there is a linear relationship for apomorphine between both efficacy and side effects. The present invention allows one to accurately target the narrow window where there is both therapeutic efficacy and an absence of significant side effects.

It is speculated that the side effects experienced by the subjects may be limited by the short exposure time, which results from the administration by inhalation. The exposure time from sublingual tablets is considerably longer, as will also be the case for oral and nasal administration.

The initial drug distribution phase extends between approximately 1 and 15 minutes after the dose is administered, with a linear elimination phase being observed over the remaining sampling time points

The pharmacokinetic profile indicates highly efficient and reproducible delivery of apomorphine via inhalation when compared to Uprirma® with a significantly higher Cₘₐₓ for any given dose of the inhaled apomorphine, very rapid absorption, as indicated by tₘₐₓ and no prolonged clearance of apomorphine with any of the inhaled doses.

The results provide validation of the predicted rapid absorption, rapid systemic availability and rapid elimination accompanied by low intra- and inter-subject plasma concentration variability plasma via inhalation mode of administration. Tolerability and the pharmacokinetic parameters from this study indicate that delivery of apomorphine by inhalation facilitates attaining the therapeutic window for apomorphine when seeking to treat erectile dysfunction.

### Example 16

### Solution pMDI Formulations

A pMDI formulation was prepared with the ingredients listed in the following table. The formulation can be placed in a 3M coated (Dupont 3200 200) canister with a Bespak BK630 series 0.22 mm actuator for subsequent delivery to the lungs of a patient as described above.

| | 200 μg Formulation | | |
|---|---|---|---|
| | Volume | Amount | Percentage |
| Apomorphine HCl | 0.0200 ml | 24 mg | 0.1931% w/w |
| (Ex. 2) | | | |
| HFA134a | 6.45 ml | 7905 mg | 63.60% w/w |
| Water | 0.75 ml | 749 mg | 6.03% |
| Ethanol | 4.75 ml | 3751.50 mg | 30.18% |
| Total Formulation | | 12429.50 mg | |
| Weight | | | |
| Total Formulation | 11.97 ml | | |
| Volume | | | |
| Estimated dose of | | 200 μg/100 μl | |
| Apomorphine HCl | | | |

It is expected that this formulation can provide a fine particle fraction of between to 10% and 30%.

### Example 17

### Suspension pMDI Formulations

Suspension pMDIs were prepared with HFA227, HFA134a, and apomorphine hydrochloride in a 3M coated (Dupont 3200 200) canister with a Bespak BK630 series 0.22 mm actuator. Specifically, the formulations set out below were prepared.

| | Formulation A | | | Formulation B | | |
|---|---|---|---|---|---|---|
| | Amount | | Percentage | Amount | | Percentage |
| Apomorphine | 26.7 | mg | 0.23% w/w | 104 | mg | 0.9% w/w |
| HCl (Ex. 2) | | | | | | |
| HFA134a | 4229 | mg | 37.14% w/w | 4321.7 | mg | 37.4% w/w |
| HFA227 | 7129.7 | | 62.62% w/w | 7129.7 | mg | 61.7% w/w |
| Total | 11385.4 | mg | | 11555.4 | mg | |
| Formulation | | | | | | |
| Weight | | | | | | |
| Total | 8.5 | ml | | 8.7 | ml | |
| Formulation | | | | | | |
| Volume | | | | | | |
| (Estimated) | | | | | | |

| | | |
|---|---|---|
| Estimated | 157 μg/50 μl | 600 μg/50 μl |
| dose of | | |
| Apomorphine | | |
| HCl | | |

Formulation B was tested with an Anderson Cascade Impactor over 10 discharges. The results were as follows, each value being an average of the 10 discharges:

| | | |
|---|---|---|
| Metered Dose | 517.43 | μg |
| Delivered Dose | 470.96 | μg |
| MMAD | 3.47 | μm |
| Fine Particle Dose | 314.140 | μg |

| | |
|---|---|
| Fine Particle Fraction | 66.7% |

wherein a fine particle is defined as a particle having a diameter of less than or equal to 5 μm.

### Example 18

### 400 μg apomorphine hydrochloride Capsule for use in Cyclohaler

Five 400 μg apomorphine hydrochloride capsules were prepared and tested in a Cyclohaler inhaler™ (available from Miat) in an ACI (U.S.P. 26, Chapter 601, Apparatus 3) configured for operation at 100 l.min⁻¹. Each capsule had a fill weight of 25 mg, and included the following components:

| Component | Weight (g) | Weight % (w/w) |
|---|---|---|
| Pharmatose 150 M | 127.725 | 85.15 |
| (DMV Pharma) | | |
| Sorbolac 400 | 12.375 | 8.25 |
| (Meggle Pharma) | | |
| Micronised Leucine | 7.500 | 5.00 |
| (As described in Example 10) | | |
| Apomorpbine Hydrochloride | 2.400 | 1.60 |
| (d₅₀ = 1.453 μm) | | |
| (As described in FIG. 2B) | | |

In this regard, Pharmatose 150M, available from DMV Pharma, comprises lactose with the following particle size distribution (according to DMV Pharma literature): 100% less than 315 μm, at least 85% less than 150 μm, at least 70% less than 100 μm, and at least 50% less than 45 μm. Sorbolac 400, available from Meggle Pharma comprises lactose with the following particle size distribution (according to Meggle Pharma literature): 100% less than 100 μm, at least 99% less than 63 μm, and at least 96% less than 32 μm.

Preparation of Pre-Blend

The Pharmatose, Sorbolac and leucine were layered in the mixing bowl so that the leucine was sandwiched between the Sorbolac, which in turn was sandwiched between the Pharmatose. The powders were blended for 60 seconds at 2000 rpm using the Retsch Grindomix High Shear Mixer described above. The pre-blend was rested for 1 hour before further use.

Preparation of Final Blend

The apomorphine hydrochloride was sandwiched between the pre-blend in the mixing bowl. Blending was carried out for 10 minutes at 2000 rpm using the Grindomix mixer. The blend was then passed through a 212 μm sieve.

Thereafter, the final blend was placed in capsules, each capsule having a fill weight of 25 mg. The capsules were then placed in a Cyclohaler and tested in an ACI (U.S.P. 26, Chapter 601, Apparatus 3), with the data analysed via the CITDAS described above, providing the following results:

| Delivered Dose (%) | 81% | |
|---|---|---|
| (100*Delivered Dose/Total Dose) | | |
| % Fine Particle Fraction | 67% | |
| (percent of the delivered dose <5 μm) | | |
| % Fine Particle Dose | 55% | |
| (percent of the total dose <5 μm) | | |
| MMAD | 2.3 | μm |
| Fine Particle Dose | 220 | μg |
| % Ultrafine Particle Dose | 44% | |
| (percent of the total dose <3 μm) | | |
| Ultrafine Particle Dose | 175 | μg |

| | |
|---|---|
| Ultrafine Particle Fraction | 53% |

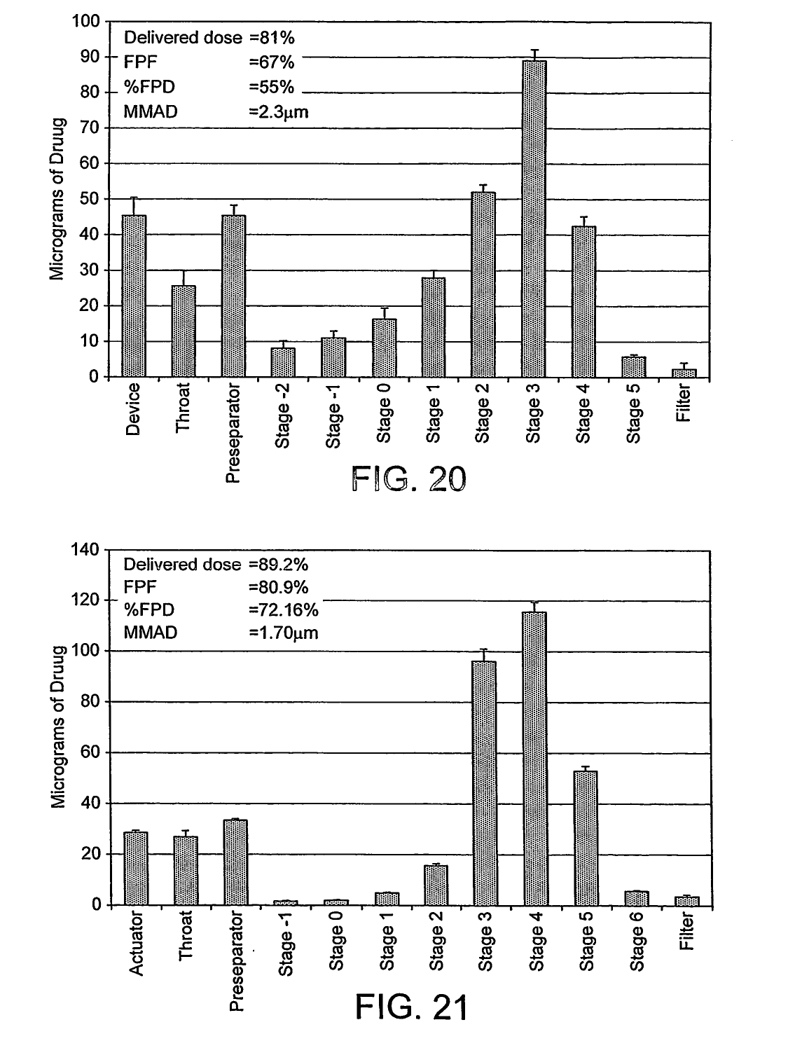

FIG. 20 illustrates the average amount (in micrograms) of drug that was delivered to each of the components of the ACI, and retained in the device. Thus, for example, the ultrafine particle dose can be produced from this data by the CITDAS package.

### Example 19

### 400 μg apomorphine hydrochloride 2 mg Blister

Five 400 μg apomorphine hydrochloride blisters were prepared and tested in the inhaler of Example 5 in an ACI (USP 26, Chapter 601, Apparatus 3) configured for operation at 60 l.min⁻¹. Each blister had a fill weight of 2 mg, and included the following components:

| Component | Weight (g) | Weight % (w/w) |
|---|---|---|
| Respitose 45-63 μm sieve | 120 | 80 |
| (As described in Example 1) | | |
| Apomorphine Hydrochloride | 30 | 20 |
| (d₅₀ = 1.453 μm) | | |
| (As described in FIG. 2B) | | |

The apomorphine hydrochloride was sandwiched between the Respitose in the mixing bowl as generally described in Examples 2(a) and 2(b). The powders were blended for 5 minutes at 2000 rpm using the Grindomix mixer. The blend was then passed through a 212 μm sieve. Thereafter, the blend was placed in blister, each blister having a fill weight of 2 mg. The blisters were then placed in the inhaler of Example 5 and tested in an ACI (U.S.P. 26, Chapter 601, Apparatus 3), with the data analysed via the CITDAS described above, providing the following results:

| | |
|---|---|
| Delivered Dose (%) | 89% |
| (100 * Delivered Dose/Total Dose) | |
| % Fine Particle Fraction | 81% |
| (percent of the delivered dose <5 μm) | |
| % Fine Particle Dose | 72% |
| (percent of the total dose <5 μm) | |
| MMAD | 1.70 μm |
| Fine Particle Dose | 288 μg |
| % Ultrafine Particle Dose | 67% |
| (percent of the total dose <3 μm) | |
| Ultrafine Particle Dose | 266 μg |
| % Ultrafine Particle Fraction | 75% |
| (percent of the delivered dose <3 μm) | |

FIG. 21 illustrates the average amount (in micrograms) of drug that was delivered to the components of the ACI, and left in the device. Thus, for example, the ultrafine particle dose can be produced from this data using the CITDAS package.

It should be noted that the MMAD of 1.70 μm generated from the ACI data is remarkably fine, and very close to the median diameter determined by laser light diffraction, for this batch of apomorphine hydrochloride (1.453 μm as reported FIG. 5B). This indicates that the inhaler is efficiently reducing the drug to, or close to, its primary particles, rather than as agglomerate. This is highly unusual for an inhaler. For example, when the same batch of apomorphine hydrochloride (i.e., in particle size) was delivered with the Cyclohaler of Example 18, a larger MMAD of 2.3 μm was measured, indicating that this formulation and device was not as efficient at eliminating agglomerates.

When compared with the formulation and inhaler of Example 18, the formulation and inhaler of Example 19 also provided a superior delivered dose (89.2% vs. 81%), fine particle fraction (81% vs. 67%), %fine particle dose (72% vs. 55%) and % ultrafine particle dose (67% vs. 44%).

It is also apparent from the above data that the formulation and inhaler of Example 19 produces an ultrafine particle fraction (<3 μm) of more than 70%. While a fine particle fraction (<5 μm) can be considered acceptable for local delivery, it is believed that for systemic delivery, even finer particles are needed, because the drug must reach the alveoli to be absorbed into the bloodstream. As such an ultrafine particle fraction in excess of 70% is particularly advantageous.

The above referenced data indicates that the preferred inhaler in accordance with the present invention is particularly efficient when combined with the preferred formulation in accordance with the present invention.

It should also be noted that both the formulation of Example 18 (with the Cyclohaler) and the formulation of Example 19 (with the preferred inhaler), provide significantly better performance than the suspension pMDI of Example 16, which had an MMAD of 3.47, an FPF of 66.7, and a % Fine Particle Dose of 52.4%.

### Example 20

### Comparison of Co-Jet Milled and Mechanofused apomorphine Formulations

A number of apomorphine hydrochloride formulations with fine excipient particles were prepared by co-jet milling and by MechanoFusion and these formulations were then tested. The co-jet milling was carried out in a jet mill, whilst the MechanoFusion process was carried out in a MechanoFusion system (Hosokawa Micron Ltd).

19.0 g of Sorbolac 400 lactose and 1.0 g of micronised L-leucine were combined in the MechanoFusion system. The material was processed at a setting of 20% power for 5 minutes, followed by a setting of 80% power for 10 minutes. This material was recovered and recorded as “2A”.

15.0 g of apomorphine hydrochloride and 0.75 g of micronised L-leucine were combined in the MechanoFusion system. The material was processed at a setting of 20% power for 5 minutes, followed by a setting of 80% power for 10 minutes. This material was recovered and recorded as “2B”.

2.1 g “2B” plus 0.4 g micronised leucine were blended by hand in a mortar and pestle for 2 minutes. 2.5 g micronised lactose was added and blended for a further 2 minutes. 5 g micronised lactose was added and blended for another 2 minutes. This mixture was then processed in the AS50 Spiral jet mill using an inlet pressure of 7 bar and a grinding pressure of 5 bar, feed rate 5 ml/min. This powder was gently pushed through a 300 μm metal sieve with a spatula. This material was recorded as “10A”.

1.5 g “10A” was combined with 0.20 g micronised L-leucine and 3.75 g of Sorbolac 400 lactose by hand in a mortar with a spatula for 10 minutes. This powder was gently pushed through a 300 μm metal sieve with a spatula. This material was recorded as “10B”.

9 g micronised apomorphine HCl plus 1 g micronised leucine were placed in the MechanoFusion system and processed at 20% (1000 rpm) for 5 minutes. This initial blend was then processed in the AS50 Spiral jet mill using an inlet pressure of 7 bar and a grinding pressure of 5 bar, feed rate 5 ml/min. This material was recorded as “11A”.

After blending, this powder was rested overnight, and then was gently passed through a 300 μm metal sieve by shaking. This material was recorded as “11B”.

2 g micronised apomorphine HCl plus 0.5 g micronised leucine were blended by hand in mortar and pestle for 2 minutes. 2.5 g micronised lactose was added and blended for a further 2 minutes. Then 5 g micronised lactose was added and blended for another 2 minutes. This mixture was then processed in the AS50 Spiral jet mill using an inlet pressure of 7 bar and a grinding pressure of 5 bar, feed rate 5 ml/min. This powder was gently pushed through a 300 μm metal sieve with a spatula. This material was recorded as “12A”.

16.5 g of Sorbolac 400 and 0.85 g of micronised leucine were placed in the MechanoFusion system and processed at 20% (1000 rpm) for 5 minutes then at 80% (400 rpm) for 10 minutes. This material was recorded as “13A”.

0.5 g micronised apomorphine HCl plus 2.0 g “13A” were blended by hand in a mortar with a spatula for 10 minutes. This powder was gently pushed through a 300 μm metal sieve with a spatula. This material was recorded as “13B”.

A number of foil blisters were filled with approximately 2 mg of the following formulations:

10A—20% apomorphine HCl, 5% 1-leucine, 75% micronised lactose (co-jet milled)

10C—26.2% apomorphine HCl, 5% 1-leucine, 68.7% sorbolac (geometric)

11B—95% apomorphine HCl, 5% 1-leucine (co-jet milled)

12A—20% apomorphine HCl, 5% leucine, 75% micronised lactose (all co-jet milled)

13B—20% apomorphine HCl, 5% 1-leucine, 75% Sorbolac 400 (leucine & Sorbolac MechanoFused)

These were then fired from an Aspirair device into an NGI at a flow rate of 60 l/m. The Aspirair was operated with a reservoir of 15 ml at 1.5 bar. Each in vitro test was conducted once to screen, and then the selected candidates were repeated. Further candidates were also repeated in ACI at 60 l/m.

TABLE 14

| Formulation | | | | |
|---|---|---|---|---|
| 2 mg, 1.5 bar | | | | |
| 15 ml reservoir | | | FPD (<5 μm) | |
| 60 l/min | MD (μg) | DD (μg) | (μg) | MMAD |
| 10A | 384 | 356 | 329 | 1.78 |
| 13B | 359 | 327 | 200 | 1.54 |
| | (1793) | (1635) | (1000) | |
| 10C | 523 | 492 | 374 | 1.63 |
| 11B | 1891 | 1680 | 1614 | 1.36 |
| | 1882 | 1622 | 1551 | 1.44 |
| | 1941 | 1669 | 1601 | 1.49 |
| Ave. | 1905 | 1657 | 1589 | 1.43 |
| SD | 32 | 31 | 33 | 0.07 |
| RSD | 1.7 | 1.9 | 2.1 | 4.6 |
| 11B | 1895 | 1559 | 1514 | 1.58 |
| | 1895 | 1549 | 1485 | 1.62 |
| | 1923 | 1565 | 1504 | 1.62 |
| ACI | | | | |
| Ave. | 1904 | 1558 | 1501 | 1.61 |
| SD | 16 | 8 | 15 | 0.02 |
| RSD | 1 | 1 | 1 | 1 |
| 12A | 414 | 387 | 363 | 1.63 |
| | 410 | 387 | 363 | 1.66 |
| | 406 | 378 | 355 | 1.68 |
| Ave. | 410 | 384 | 360 | 1.66 |
| SD | 4 | 5 | 5 | 0.03 |
| RSD | 1 | 1 | 1 | 2 |
| Total ave. | 2050 | 1920 | 1800 | |
| 12A | 395 | 365 | 341 | 1.80 |
| | 411 | 385 | 360 | 1.85 |
| | 400 | 370 | 349 | 1.84 |
| ACI | | | | |
| Ave. | 402 | 373 | 350 | 1.83 |
| SD | 8 | 10 | 10 | 0.04 |
| RSD | 2 | 3 | 3 | 2 |
| Total ave. | 2011 | 1866 | 1750 | |

TABLE 15

| Formulation | | | | | |
|---|---|---|---|---|---|
| 2 mg, 1.5 bar | FPF | FPF | FPF | | |
| 15 ml reservoir | (MD) % | (ED) % | (ED) % | FPF(ED) % | FPF(ED) % |
| 60 l/min | (<5 μm) | (<5 μm) | (<3 μm) | (<2 μm) | (<1 μm) |
| 10A | 86 | 93 | 87 | 60 | 13 |
| 13B | 56 | 61 | 52 | 42 | 19 |
| 10C | 72 | 76 | 67 | 51 | 16 |
| 11B | 85 | 96 | 95 | 81 | 24 |
| | 82 | 96 | 93 | 77 | 22 |
| | 82 | 96 | 92 | 74 | 20 |
| Ave. | 83 | 96 | 93 | 77 | 22 |
| SD | | 0 | 1.5 | 3.5 | 2 |
| RSD | | 0 | 1.6 | 4.5 | 9.1 |
| 11B | 80 | 97 | 94 | 74 | 14 |
| | 78 | 96 | 93 | 70 | 14 |
| | 78 | 96 | 94 | 72 | 12 |
| ACI | | | | | |
| Ave. | 79 | 96 | 94 | 72 | 13 |
| SD | | 1 | 1 | 2 | 1 |
| RSD | | 1 | 1 | 3 | 9 |
| 12A | 88 | 94 | 89 | 68 | 13 |
| | 89 | 94 | 89 | 66 | 12 |
| | 87 | 94 | 88 | 64 | 12 |
| Ave. | 88 | 94 | 89 | 66 | 12 |
| SD | | 0 | 1 | 2 | 1 |
| RSD | | 0 | 1 | 3 | 5 |
| 12A | 86 | 94 | 85 | 57 | 9 |
| | 88 | 93 | 84 | 55 | 8 |
| | 87 | 94 | 85 | 56 | 8 |
| ACI | | | | | |
| Ave. | 87 | 94 | 85 | 56 | 8 |
| SD | | 1 | 1 | 1 | 1 |
| RSD | | 1 | 1 | 2 | 7 |

TABLE 16

| Formulation | | | | |
|---|---|---|---|---|
| 2 mg, 1.5 bar | | | | |
| 15 ml reservoir | | | | |
| 60 l/min | Recovery | Throat | Blister | Device |
| 10A | 96% | 5% | 0.3% | 7% |
| 13B | 94% | 29% | 3% | 6% |
| 10C | 100% | 16% | 2% | 4% |
| 11B | 101% | 2% | 0.6% | 10% |
| | 99% | 2% | 0.2% | 14% |
| | 102% | 2% | 0.3% | 14% |
| Ave. | 101% | 2% | 0.4% | 13% |
| SD | 1.5 | 0 | 0.2 | 2.3 |
| RSD | 1.5 | 0 | 57 | 18 |
| 11B | 100% | 1% | 0.5% | 17% |
| | 100% | 2% | 0.1% | 18% |
| | 101% | 2% | 0.4% | 18% |
| ACI | | | | |
| Ave. | 100% | 2% | 0.3% | 18% |
| SD | 1 | 1 | 0.2 | 1 |
| RSD | 1 | 35 | 62 | 3 |
| 12A | 109% | 4% | 0.3% | 6% |
| | 108% | 4% | 0.2% | 6% |
| | 107% | 4% | 0.02% | 7% |
| Ave. | 108 | 4% | 0.2 | 6% |
| SD | 1 | 0 | 0.1 | 1 |
| RSD | 1 | 0 | 82 | 9 |
| 12A | 104% | 3% | 0.4% | 7% |
| | 108% | 4% | 0.2% | 6% |
| | 105% | 2% | 0.4% | 7% |
| ACI | | | | |
| Ave. | 106% | 3% | 0.3 | 7% |
| SD | 2 | 1 | 0.1 | 1 |
| RSD | 2 | 33 | 35 | 9 |

The co-jet milled formulations once again exhibited exceptional FPFs when it is dispensed using an active dry powder inhaler device. The improvement appears to be largely due to reduced throat deposition which was less than 5%, compared to between 16 and 29% for the MechanoFused formulations. “12A” was produced as a repeat of “10A”, but excluding the MechanoFused pre-blend (to show it was not required).

The reproducibility of the FPFs obtained with the formulation 12A, the preparation of which is described above, was tested.

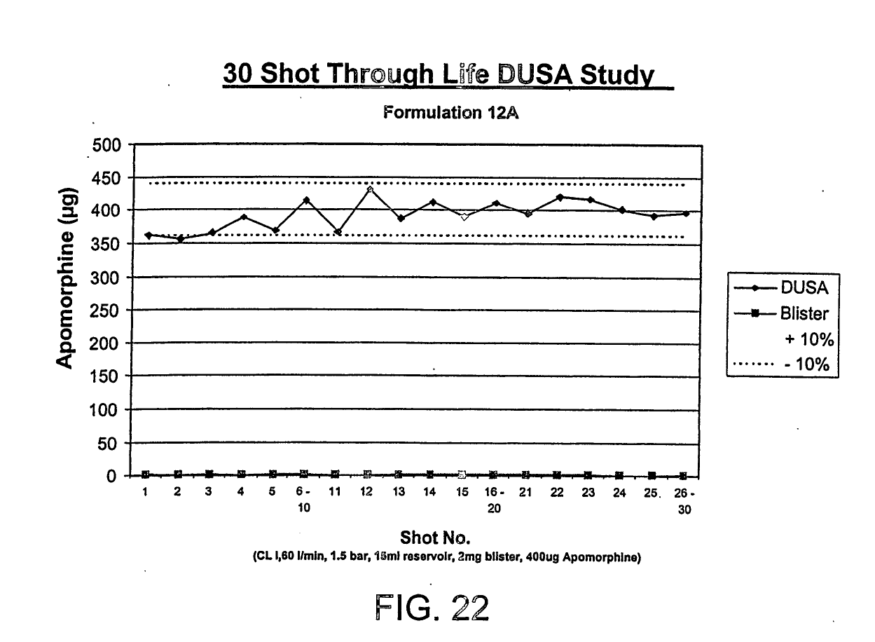

A number of foil blisters were filled with approximately 2 mg of formulation 12A. Through life dose uniformity was tested by firing 30 doses, with the emitted doses collected by DUSA. Through life dose uniformity results are presented in the graph in FIG. 22.

The mean ED was 389 μg, with an RSD of 6.1% and the through life delivery of this drug-lactose formulation was very good.

### Example 21

### Provision of Appropriate apomorphine Doses

From the phase 1 study it was discovered that the maximum tolerated dose of inhaled apomorphine was around 900 μg.

The formulation used in Example 7 incorporated 20% w/w (600 μg) of apomorphine. Experiments with blister fill weights of 3 mg were performed and these blisters were shown to provide a fine particle fraction of 72%. To obtain a 900 μg dose, it would therefore be necessary to increase the blister fill weight from 3 mg to 4.5 mg of the 600 μg drug formulation, or to use a number of blisters (e.g. 1×600 μg/3 mg and 1×300 μg/1.5 mg).

Another option would be to increase the drug load from 20% to 30% w/w to maintain a fill weight of 3 mg per blister.

This formulation can be manufactured in the manner set forth above in Example 2, with the components provided in the following amounts for a 3 mg blister:

| Composition | Amount (μg) | Percent |
|---|---|---|
| Apomorphine HCl | 900 | 30 |
| Lactose | 2100 | 70 |
| Total | 3000 | 100 |

The ACI results (set out in Table 17 below) show that that when raising the fill weight from 3 to 4.5 mg in the blister, the FPF decreases slightly using the 20% w/w formulation. The FPF of the 30% w/w formulation increased slightly to 74%. This indicates that a 30% w/w drug formulation can be used to increase dose.

TABLE 17
Summary of ACI results of 20 and 30% w/w drug formulation

| | Fine particle dose | | Fine particle dose | |
|---|---|---|---|---|
| | ≦5 μm | | ≦3 μm | |
| | | Fraction | | Fraction |
| Formulation/blister detail | Dose (μg) | (%) | Dose (μg) | (%) |
| 20% w/w 3 mg (600 μg) | 370.41 | 72.45 | 282.65 | 55.28 |
| 20% w/w 4.5 mg (900 μg) | 550.48 | 69.19 | 412.88 | 51.90 |
| 30% w/w 3 mg (900 μg) | 611.07 | 74.33 | 460.98 | 56.08 |

### Example 22

### Comparison of use of Sieved and Unsieved Lactose Carrier Particles

As part of the ongoing 30% w/w blend development a blend using Sorbolac 400 instead of Respitose SV003 was prepared.

The formulation was prepared with unsieved Sorbolac 400 and sieved Sorbolac 400 (using a 100 μm mesh sieve).

This formulation can be manufactured in the manner set forth above in Example 2, with the components provided in the following amounts for a 3 mg blister:

| Composition | Amount (μg) | Percent |
|---|---|---|
| Apomorphine HCl | 900 | 30 |
| Sorbolac 400 | 2100 | 70 |
| Total | 3000 | 100 |

Initial results show that the FPF of the sieved formulation (65%) is higher than the FPF of the unsieved formulation (61%).

### Example 23

### Preparation of pMDI Formulation

A further formulation according to the present invention may be prepared as follows. 12.0 g micronised apomorphine and 4.0 g lecithin S PC-3 (Lipoid GMBH) are weighed into a beaker. The powder is transferred to the Hosokawa AMS-MINI MechanoFusion system via a funnel attached to the largest port in the lid with the equipment running at 3.5%. The port is sealed and the cooling water switched on. The equipment is run at 20% for 5 minutes followed by 50% for 10 minutes. The equipment is switched off, dismantled and the resulting formulation recovered mechanically.

Preparation of Cans:

0.027 g powder is weighed into the can, a 50 μl valve is crimped to the can and 12.2 g HFA 134a is back filled into the can.

### Example 24

### Preparation of MechanoFused Formulation for use in Passive Device

A further formulation according to the present invention may be prepared as follows. 20 g of a mix comprising 20% micronised apomorphine, 78% Sorbolac 400 lactose and 2% magnesium stearate are weighed into the Hosokawa AMS-MINI MechanoFusion system via a funnel attached to the largest port in the lid with the equipment running at 3.5%. The port is sealed and the cooling water switched on. The equipment is run at 20% for 5 minutes followed by 80% for 10 minutes. The equipment is switched off, dismantled and the tesulting formulation recovered mechanically.

### Example 24

### Apomorphine Free Base Formulation

A 600 microgram formulation can be manufactured in the manner set forth above with regard to Example 2, with the components provided in the following amounts:

| Composition | Amount (μg) | Percent |
|---|---|---|
| Apomorphine free base | 600 | 20 |
| Lactose | 2400 | 80 |
| Total | 3000 | 100 |

In the preceding specification, the invention has been described with reference to specific exemplary embodiments and examples thereof. It will, however, be evident that various modifications and changes may be made thereto without departing from the broader spirit and scope of the invention as set forth in the claims that follow. The specification and drawings are accordingly to be regarded in an illustrative manner rather than a restrictive sense.

1. A composition for treating sexual dysfunction by pulmonary inhalation, said composition comprising apomorphine, the apomorphine being in the form of a free base, pharmaceutically acceptable salt or ester.

2. A composition as claimed in claim 1, wherein the apomorphine is apomorphine hydrochloride.

3. A composition as claimed in claim 1, wherein the administration of the composition by pulmonary inhalation provides a Cₘₐₓ within 1 to 5 minutes of administration.

4. A composition as claimed in claim 3, wherein the Cₘₐₓ is at least 2 ng/ml.

5. A composition as claimed in claim 4, wherein the Cₘₐₓ is at least 7 ng/ml.

6. A composition as claimed in claim 1, wherein the administration of the composition by pulmonary inhalation provides a terminal elimination half-life of between 50 and 70 minutes.

7. A composition as claimed in claim 1, wherein the administration of the composition by pulmonary inhalation provides a dose dependent AUC_{0-∞}.

8. A composition as claimed in claim 1, wherein the administration of the composition by pulmonary inhalation provides a dose dependent AUC₀₋ₜ.

9. A composition as claimed in claim 1, wherein the administration of the composition by pulmonary inhalation provides a dose dependent Cₘₐₓ.

10. A composition as claimed in claim 1, wherein the administration of the composition by pulmonary inhalation is not accompanied with the adverse side effects usually associated with the administration of apomorphine.

11. A composition as claimed in claim 1, wherein the composition provides a dose of apomorphine of from about 100 to about 1600 micrograms of apomorphine or a pharmaceutically acceptable salt or ester thereof (based on the weight of the hydrochloride salt).

12. A composition as claimed in claim 11, wherein the dose is from about 200 to about 1600 micrograms.

13. A composition as claimed in claim 12, wherein the dose is from about 300 to about 1200 micrograms.

14. A composition as claimed in claim 13, wherein the dose is from about 400 to about 1000 micrograms.

15. A composition as claimed in claim 1, wherein the sexual dysfunction is erectile dysfunction.

16. A composition as claimed in claim 1, wherein the sexual dysfunction is female sexual dysfunction.

17. A composition as claimed in claim 15, wherein the erectile dysfunction is psychogenic.

18. A composition as claimed in claim 15, wherein the erectile dysfunction is organic.

19. A composition as claimed in claim 1, wherein the composition is a dry powder composition.

20. A composition as claimed in claim 19, wherein the apomorphine has a mass median aerodynamic diameter of 10 μm or less.

21. A composition as claimed in claim 20, wherein the mass median aerodynamic diameter is 5 μm or less.

22. A composition as claimed in claim 19, wherein at least 90% of the apomorphine has a particle size of 10 μm or less.

23. A composition as claimed in claim 22, wherein at least 90% of the apomorphine has a particle size of 5 μm or less.

24. A composition as claimed in claim 19, wherein the composition further comprises an additive material.

25. A composition as claimed in claim 24, wherein the additive material is provided in an amount from about 0.15% to about 5% of the composition, by weight.

26. A composition as claimed in claim 24, wherein the additive material is selected from the group consisting of leucine, magnesium stearate, lecithin, and sodium stearyl fumarate.

27. A composition as claimed in claim 19, wherein the composition further comprises an excipient material.

28. A composition as claimed in claim 27, wherein the excipient material is in the form of carrier particles having an average particle size of 40 to 70 μm.

29. A composition as claimed in claim 1, wherein the composition comprises a solution pMDI formulation including a propellant, a solvent and water.

30. A composition as claimed in claim 29, wherein the propellant is HFA134a and/or HFA227.

31. A composition as claimed in claim 29, wherein the solvent is ethanol.

32. A composition as claimed in claim 29, wherein said water is present in an amount from greater than 2% by weight to about 10% by weight of the solution pMDI formulation.

33. A composition as claimed in claim 1, wherein the composition is a suspension pMDI formulation including a propellant.

34. A composition as claimed in claim 33, wherein the propellant is HFA134a and/or HFA227.

35. A composition as claimed in claim 34, wherein the propellant includes about 60% by weight HFA134a and about 40% by weight HFA227.

36. A method of treating sexual dysfunction, the method comprising administering to a subject in need of such treatment a composition as claimed in claim 1.

37. A method as claimed in claim 36, wherein the sexual dysfunction is male erectile dysfunction.

38. A method as claimed in claim 36, wherein the sexual dysfunction is female sexual dysfunction.

39. A method as claimed in claim 36, wherein the method does not cause the adverse side effects normally associated with the administration of apomorphine.

40-41. (canceled)

42. A dry powder inhaler device comprising a composition as claimed in claim 1.

43. A dry powder inhaler device as claimed in claim 42, wherein the inhaler is an active inhaler.

44. A dry powder inhaler as claimed in claim 42, wherein the inhaler is a breath actuated inhaler device.

45. A blister for use in a dry powder inhaler device as claimed claim 42, wherein the blister contains the composition.

46. A blister as claimed in claim 45, wherein the blister is a foil blister.

47. A blister as claimed in claim 45, wherein the blister comprises polyvinyl chloride or polypropylene in contact with the composition.
